(19) United States
(12) Patent Application Publication
(10) Pub. No.: US 2024/0408302 A1
(43) Pub. Date: Dec. 12, 2024

(54) CLOSED-LOOP SODIUM ADMINISTRATION FOR TREATMENT OF CEREBRAL EDEMA

(71) Applicant: Naval Medical Research Command, Silver Spring, MD (US)

(72) Inventors: Geoff Peitz, San Antonio, TX (US); William R. D'Angelo, San Antonio, TX (US)

(21) Appl. No.: 18/117,885

(22) Filed: Mar. 6, 2023

Related U.S. Application Data
(60) Provisional application No. 63/317,042, filed on Mar. 6, 2022.

(51) Int. Cl. A61M 5/168; A61K 9/00; A61K 33/14; A61M 5/142

(52) U.S. Cl. CPC A61M 5/16831; A61K 9/0019; A61K 33/14; A61M 5/142; A61M 2205/18; A61M 2205/3334; A61M 2230/20

(57) ABSTRACT

A hypertonic saline (HTS) administration system comprising a sodium sensor for continuous monitoring of sodium concentration of a patient at a predetermined frequency, a infusion pump for delivery of HTS to the patient and a controller for adjusting the infusion rate of an infusion pump based on real-time measurement of sodium concentration of the patient. The sodium sensor comprises a sensor cable for placement through a catheter to position a distal sensor tip at a selected in vivo sensor site. A proximal end of the sensor cable seats within a connector fitting mounted on the catheter at a convenient and accessible subcutaneous position. The connector fitting couples the sensor cable to the controller which adjusts the infusion rate of the infusion pump.

### CROSS REFERENCE TO RELATED APPLICATIONS

This application claims priority to U.S. Provisional Application No. 63/317,042, filed Mar. 6, 2022, which is hereby incorporated by reference.

### TECHNICAL FIELD

This invention relates to a method, a system and a device for treating cerebral edema or hyponatremia by closed-loop sodium monitoring and administration. In particular, this invention relates to a controller for intravenous hypertonic saline (HTS) administration using an infusion pump.

### BACKGROUND

Traumatic brain injury (TBI) occurs over 2.8 million times per year in the United States, causing tens of thousands of death per year.⁷ While global data are mixed, TBI is among the leading causes of death and disability worldwide, occurring in an estimated 69 million people per year worldwide.^{8, 9} Besides surgical evacuation of hematomas, acute TBI treatment is heavily focused on reducing cerebral edema and intracranial pressure (ICP) to mitigate secondary brain injury. Hyperosmolar agents such as hypertonic saline (HTS) or mannitol are mainstays of ICP management. The benefits include reduction in edema, improvement in cerebral perfusion, and possible modulation of the inflammatory response through alteration of gene expression.¹⁰⁻¹⁶ Many institutions have established guidelines for the treatment of cerebral edema with hypertonic saline bolus doses and continuous infusion rates using estimated change in plasma sodium concentration based on a patient's body water content.¹⁷ However, these guidelines fail to account for varying sodium excretion rates, sodium input sources besides the administration of HTS, and changes in body water content. Frequent blood sampling for sodium concentration measurement and monitoring by a physician is required to ensure that the sodium concentration rises as expected in response to HTS treatment, and then remains in a tight therapeutic window of homeostasis. If the plasma sodium concentration rises too quickly, it can cause non-anion gap metabolic acidosis or a serious neurological condition called osmotic demyelination syndrome.³ If it rises too slowly, dehydration can persist, and in TBI, cerebral edema can build up quickly and cause cerebral herniation syndrome. If plasma sodium concentration is lowered too quickly, it can cause rebound cerebral edema, and cerebral herniation syndrome (especially in pediatrics). As shown in FIG. 1, even with frequent blood draws for plasma sodium measurement, a patient's sodium concentration can vary greatly over time, and it can take many hours to reach the target with traditional HTS administration.

The current standard method for administration of HTS involves calculating the expected volume of HTS needed to increase the plasma sodium concentration based on a patient's weight and the patient's current plasma sodium concentration. However, additional sodium sources and varying renal excretion of sodium makes the calculated dose unreliable, so the patient's plasma sodium concentration is checked intermittently. The nurse draws blood and sends it to the lab. The lab processes the blood and records the sodium concentration in the chart. Then, the physician views the lab results and orders a new HTS infusion rate. Finally, the nurse adjusts the infusion pump according to the new order. The process is repeated at intervals of 4-6 hours until the patient's plasma sodium concentration is within the therapeutic range. The entire process is slow and labor intensive and does not correspond to real-time changes in plasma sodium level.

Therefore, there is an urgent need for more accurate HTS administration in treatment of cerebral edema and intracranial hypertension. This invention describes a closed-loop control algorithm for an infusion pump used in intravenous hypertonic saline (HTS) administration, which adjusts the rate of HTS administration based on continuous sodium concentration monitoring to treat hyponatremia in general or cerebral edema and intracranial hypertension in patients with brain injuries. A closed-loop system for HTS administration can alleviate many issues with the traditional HTS administration, by rapidly adjusting HTS based on real-time feedback of patient's sodium levels.

### SUMMARY OF THE INVENTION

In accordance with the invention, an infusion pump system includes an infusion pump for programmed operation to deliver a hypertonic saline (HTS) to a patient, in combination with a sodium sensor for closed loop control of pump operation. In the preferred form, the infusion pump is controlled automatically in response to sodium concentration measurements, by means of a direct or telemetric coupling with the sensor. The sodium sensor may be anchored within the patient by a subcutaneously mounted and easily accessed connector fitting having means for coupling or relaying sensor signals to the infusion pump. Alternatively, the sodium sensor is capable of non-invasive continuous monitoring of plasma or interstitial sodium concentration measurement. In a preferred embodiment, sodium sensors have precision within I millimole per liter (mmol/L) in the range of 120-170 mmol/L and a sampling time of 60 seconds or less.

A preferred connector fitting has a generally cylindrical configuration adapted for convenient mounting beneath the patient's skin at a proximal end of a catheter leading to a selected in vivo sensor site. The sodium sensor comprises an elongated sensor cable for placement through the connector fitting, and catheter to position a sensor tip at a distal end of the cable substantially at the sensor site. A proximal end of the sensor cable seats within the connector fitting and includes means such as contacts or the like for coupling with the connector fitting so that the connector fitting provides means for electrically coupling the sensor cable to the pump system.

Another aspect of this invention is a controller for intravenous hypertonic saline (HTS) administration to be used in combination with an infusion pump and a sodium sensor, whereas the controller automatically adjusts the infusion rate of HTS, based on real-time sodium concentration measurements received from the sodium sensor at a predetermined sampling rate.

Yet another aspect of this invention is an algorithm used by a standalone controller used in combination with an infusing pump or the controller of an infusing pump. The algorithm is used to automatically administer HTS to a patient based on real-time measured plasma sodium concentration of the patient.

Other features and advantages of the present invention will become more apparent from the following detailed description, taken in conjunction with the accompanying drawings, which illustrate, by way of example, the principles of the invention.

### DESCRIPTION OF DRAWINGS

The accompanying drawings illustrate the invention. In such drawings:

FIG. 1 illustrates the changes in plasma sodium concentration over time in a patient with severe traumatic brain injury, who is treated with both boluses and continuous infusion of hypertonic saline towards a goal sodium range of 150-155 mmol/L to reduce cerebral edema. The patient was treated with the current standard practice involving blood samples sent to the lab for sodium concentration measurement every 4-6 hours and subsequent adjustment of the hypertonic saline infusion by the physician based on the lab results.

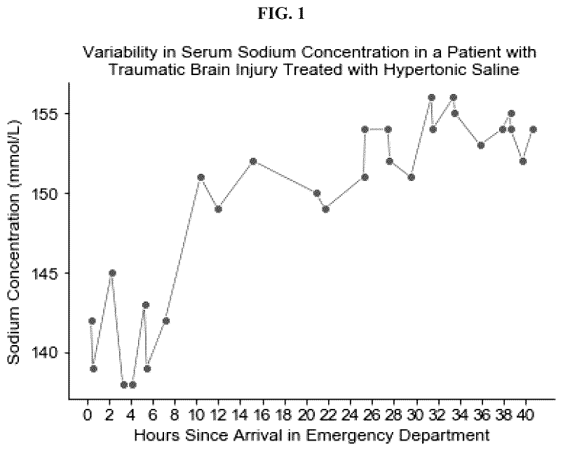

FIG. 2 shows the calibration curve for an electrical conductivity meter. Data points are electrical conductivity in millisiemens per centimeter (mS/cm) at discrete saline concentrations in millimoles per liter (mmol/L). The line is an ordinary least squares linear regression with saline concentration (mmol/L) as the independent variable and conductivity (mS/cm) as the dependent variable, and the equation for this line is shown to the bottom right. To the upper left is the R-squared value of 0.970, indicating a good fit.

FIG. 3 is a scatter plot showing the relationship between saline concentration in millimoles per liter (mmol/L, circles, left vertical axis) in a beaker as measured by an electrical conductivity meter and the hypertonic saline infusion pump stepper motor speed in steps per second (triangles, right vertical axis), as determined by the proportional-integral-derivative control program over time. At 160 mmol/L, the speed changes to 0, and the pump motor stops because target sodium concentration has been reached.

FIG. 4 is a calibration plot for converting sodium ion (Na+) selective electrode

(ISE) measurements to sodium concentration. ISE measurements in millivolts (m V) are on the horizontal axis, and Na+ concentrations in millimoles per liter (mmol/L) are on the vertical axis which has a natural logarithmic scale (base e). The dashed line is an ordinary least squares linear regression with m V as the independent variable and mmol/L as the dependent variable. To the bottom right are the regression equation, and the associated R-squared value of 0.998, indicating a good fit.

FIG. 5 shows data from an in vitro proof of concept experiment of the invention. Porcine blood was continuously stirred in a small vial while peristaltic pumps circulated the blood via tubing through a small open container equipped with a sodium ion (Na+) selective electrode (ISE). The ISE measured the plasma sodium concentration in millimoles per liter (mmol/L, circles, left vertical axis) every 60 seconds. A microcontroller read the data and calculated a new hypertonic saline (HTS) infusion rate (mL/min, triangles, right vertical axis) according to the proportional-integral-derivative control program. Time in minutes (min) is shown on the horizontal axis. At 13.5 minutes, the plasm sodium concentration reached the target of 155 mmol/L, and thus, the HTS infusion stopped (flow=0 mL/min).

FIG. 6 is a system diagram illustrating how controller/control unit 13 adjusts the rate of HTS administration (infusion rate) based on continuous monitoring of a patient's plasma sodium concentration.

FIG. 7 is a diagram showing an alternative system set up, in which the controller/control unit 13 adjusts the rate of HTS administration (infusion rate) based on continuous interstitial sodium concentration monitoring such as with a transdermal sensor.

FIG. 8 is a logic diagram showing an algorithm used for calculating the rate of hypertonic saline (HTS) infusion using a proportion-integral-derivative (PID) mechanism.

FIG. 9A shows the experimental setup for in vivo testing of this invention in a rat model as described in Example 3. Catheters from the femoral artery and vein, along with a peristaltic pump and tubing, form an extracorporeal circuit which pumps blood through a flow-through sodium ion selective electrode (ISE). The ISE measurements are transmitted to the microcontroller that controls the rate of hypertonic saline administration into the venous catheter. Na+ ISE is sodium ion selective electrode.

FIG. 9B shows a photo of the experimental setup for shows a photo of the experimental setup used for in vivo testing of the instant invention in a rat model as described in Example 3.

FIG. 10 shows schematic of a closed loop infusion system to treat cerebral edema or intracranial hypertension via HTS administration with calibration of the sodium ISE using pinch valves and a reservoir of standard solution with a known sodium concentration.

FIG. 11 is a photo of experimental set up as described in Example 2

FIG. 12 shows the sequence of interactions between the microcontroller, infusion pump, and ion-specific electrode (ISE) meter for in vivo administration of HTS experiment in a rat model. The infusion pump adds hypertonic saline (HTS) to the patient's intravascular space (1), increasing the plasma sodium content. Water and sodium added to the intravascular space in intravenous fluid or medication infusions or absorbed enterally also change the patient's plasma sodium concentration (2). The patient's blood flows through a circulation pump (3), which maintains flow through the ISE (4). The circulation pump speed can be controlled by a rotary encoder to adjust the flow. The microcontroller sends the sodium measurement command to the ISE meter (5), which measures the voltage from a sodium ISE in contact with the patient's blood. The microcontroller reads the voltage measurement from the ISE meter (5) and converts the voltage to sodium concentration based on previous calibration. The microcontroller then calculates the cumulative volume of HTS administered based on a revolution counter in the infusion pump (7). Finally, microcontroller calculates a new rate for HTS infusion and sends a command the infusion pump to change the rate of HTS infusion (8). Every 30 seconds, the ISE measures a new plasma sodium concentration which the microcontroller uses to calculate a new HTS infusion rate. Every 10 minutes, the microcontroller sends a signal to a switch board close Pinch Valve I and open Pinch Valve 2 (9). This interrupts circulation of blood through the ISE and allows the circulation pump to move the standard Na+ solution for calibration through the ISE (10). The microcontroller then performs a one-point recalibration, and the pinch valves reset to allow blood to again flow through the ISE. The 10-minute calibration period may be adjusted as the user desires.

FIG. 13A shows logic diagram for the initiation algorithm that sets the initial rate for HTS administration. FIG. 13A-D show the logic diagram for the present invention which uses a patient's plasma sodium concentration measurement to determine the rate of hypertonic saline administration (HTS). This is the treatment algorithm that was tested in the rat model experiments described in Example 3.

FIG. 13B shows the main algorithm for adjusting the rate of HTS administration to reach the goal sodium concentration. FIG. 13A-D show the logic diagram for the treatment algorithm of the present invention which uses a patient's plasma sodium concentration measurement to determine the rate of hypertonic saline administration (HTS). This is the treatment algorithm that was tested in the rat model experiments described in Example 3.

FIG. 13C shows the overshoot algorithm in case of real sodium concentration surpassing the intended sodium concentration. FIG. 13A-D show the logic diagram for the treatment algorithm of present invention

FIG. 13D shows the maintenance algorithm for maintaining a stable sodium concentration when it reaches the goal sodium concentration. FIG. 13A-D show the logic diagram for treatment algorithm of the present invention.

FIG. 14 shows adjustment of sodium concentration in a saline solution in vitro using the closed-loop infusion system. The starting sodium concentration in the solution was 132 mmol/L, and the starting volume was 15 mL. HTS was infused as the solution circulated through the flow-through sodium ISE, and the microcontroller adjusted the HTS infusion rate according to the sodium concentration measured every 30 seconds, as programmed. The intended sodium concentration is represented by the dot-dash line that rises linearly by 10 mmol/L over 20 minutes. Dots represent ISE-measured sodium concentration (mmol/L), and triangles represent HTS infusion rate (mL/min). ISE=ion-selective electrode, HTS=hypertonic saline.

FIG. 15 shows the ISE sodium concentration (lines with dots) and HTS infusion rate (lines with triangles) over time for the rats from the experimental group (left column) and control group (right column) with the lowest (top row) and highest (bottom row) sum of errors between actual and intended sodium concentrations. Sodium concentration measurements from the benchtop analyzer (benchmark sodium concentrations) are marked by diamonds at the time each blood sample was drawn. Vertical, green, dashed lines mark the timepoints at which these benchtop analyzer measurements were used to recalibrate the ISE. Black vertical lines mark the start of the HTS infusion. ISE=ion selective electrode, HTS=hypertonic saline.

### DESCRIPTION OF THE INVENTION

A closed loop sodium administration system for the treatment of cerebral edema, intracranial hypertension and/or hyponatremia has been developed, which is used in conjunction with a patient's intravenous hypertonic saline (HTS) administration to account for varying sodium excretion rates, additional sodium input sources besides the administration of HTS, and changes in patent's body water content. The disclosed system in an exemplary embodiment, adjusts the rate of HTS administration based on continuous real-time measurements of patient sodium concentration at a predetermined rate.

Although closed-loop administration system is established for other drugs such as anesthetics and insulin, its application to sodium is novel, and no system currently exists that measures plasma sodium concentration of a patient in real time and adjust HTS infusion rate accordingly.

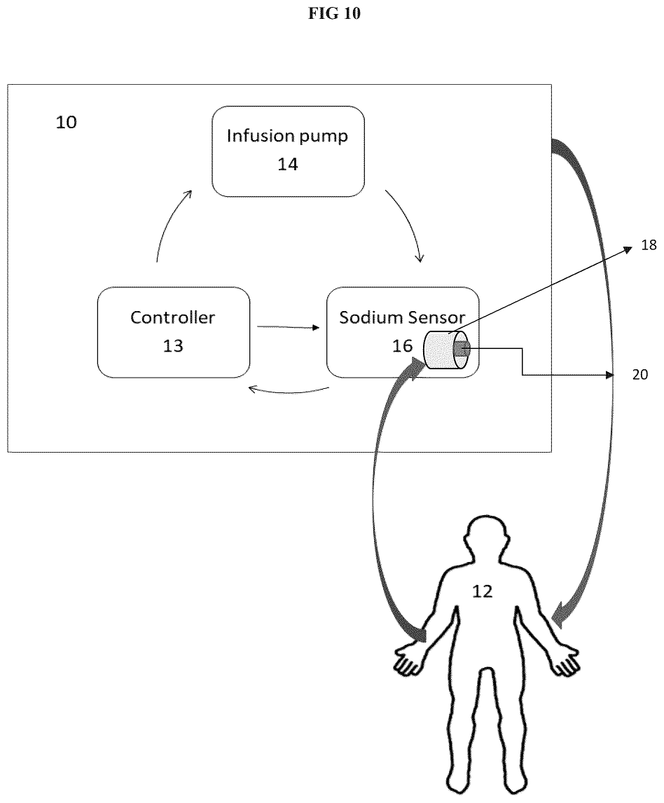

As shown in the exemplary drawings, a closed loop sodium administration system referred to generally in FIG. 10 by the reference numeral 10 is provided for delivering accurately controlled HTS to a patient 12, which can act as a stand-alone unit or part of an infusion pump. The closed loop sodium administration system 10 generally comprises an infusion pump 14, which responds to control signals from control unit or controller 13, which controls the infusion of HTS to a patient based on continuous measurement of patient sodium concentration using a sensor 16. The control unit can adjust the infusion rate of the infusion pump based on real-time patient sodium concentration measurements. In accordance with a primary aspect of the invention, the sensor unit 16 may further include a subcutaneously mounted connector fitting 18 for anchoring an in vivo sodium sensor 20. Alternatively, the sodium sensor is a non-invasive remote sodium sensor.

The improved infusion pump system 10 of the present invention continuously monitors patient's sodium concentration on a predetermined sampling frequency or rate and controls the operation of the infusion pump 14 based on a combination of factors, such as measured sodium concentration, cumulative infusion volume and current infusion rate. The HTS infusion to a patient thus may be administered in response to actual patient's need as reflected in real-time sodium concentration measurement. In an embodiment, a connector fitting 18 provides a relatively simple and easily accessed structure for coupling the sodium sensor 20 with other system components, while permitting access to the sodium sensor for removal and replacement. In this regard, the sodium sensor 20 can be replaced on a periodic basis, typically at the conclusion of a service life, while permitting the remaining system components to remain undisturbed within the patient.

In a preferred system arrangement as shown in FIG. 10, an infusion pump 14 is provided for automatically delivering fluids, medications, or diagnostic substances within the patient at a variable infusion rate, based on pre-set rate and/or correction signals received from the control unit/controller 13, which controls the operation of the infusion pump. Although numerous types of regulated infusion pumps can be used, a typical infusion pump comprises an intravenous fluid source having suitable infusion fluid such as HTS, which feeds into an infusion pump 14 that delivers infusion fluid to a patient at a controlled rate of flow, by forcing it through the IV catheter. The infusion pump used in connection with the subject invention may also include a bubble detector, error-empty bottle detector and alarms useful in the present system. Interface between the infusion pump 14 and the controller/control unit 13 can be any of the common wired or wireless connections.

The controller/control unit 13 is suitably programmed and operated to deliver the fluid to a patient, such as HTS, in accordance with individual patient need, including but not limited to closed loop response to sodium concentration measurement as will be described in more detail later. The controller/control unit 13 controls the sampling frequency or rate of the sodium sensor, receives signals from the sodium sensor and determines the rate of infusion based on these sodium measurements and controls the operation of the infusion pump based on the treatment algorithm. For example, a patient's sodium concentration can be measured at rate of every 30-60 seconds. The rate of infusion pump is controlled automatically in response to sodium concentration measurements, by means of a direct or telemetric coupling with the sensor via the control unit.

The sodium sensor of the subject closed loop HTS administration system monitors the patient's real-time sodium concentration continuously or intermittently. The infusion system 10 provides the appropriate therapy to the patient by modification of infusion rates of HTS in response to a correction signal from the controller 13 to the infusion pump 14, which is based on real-time sodium measurements and a treatment algorithm. Examples of a sodium sensor may include but not limited to an ion selective electrode (ISE) meter, sodium-selective optode or near-infrared spectrometer. In a preferred embodiment, the sodium sensor must be accurate within 1 mmol/L in the range of 120-170 mmol/L and have a sampling rate of 60 seconds or less.

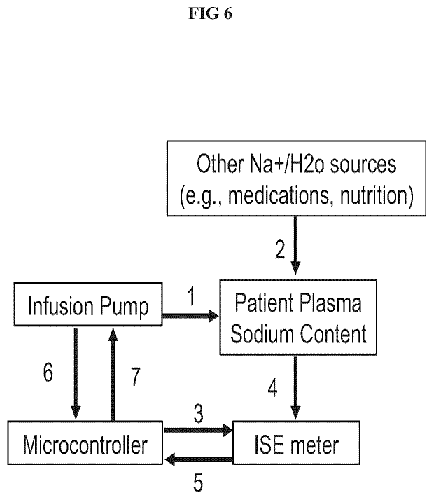

FIG. 6 shows the sequence of interactions between the controller, infusion pump, and ISE meter in operation. The infusion pump adds hypertonic saline (HTS) to the patient's intravascular space (1), increasing the plasma sodium content. Water and sodium added to the intravascular space in intravenous fluid or medication infusions and absorbed enterally also change the patient's plasma sodium concentration (2). The controller sends the sodium measurement command to the ISE meter (3), which measures the voltage from a sodium ISE indwelling in the patient's blood (4). The controller reads the voltage measurement from the ISE meter (5) and converts the voltage measurement to real time sodium concentration based on calibration. The controller then calculates the cumulative volume of HTS administered based on a revolution counter in the infusion pump (6). Finally, the controller calculates a new infusion rate of HTS infusion and sends a command signal to the infusion pump to change the rate of HTS infusion (7). Each minute, the ISE measures a new plasma sodium concentration, which the controller uses to calculate a new HTS infusion rate and controls the operation of the infusion pump using a treatment algorithm, which delivers HTS to the Patient based on command signal from the controller.

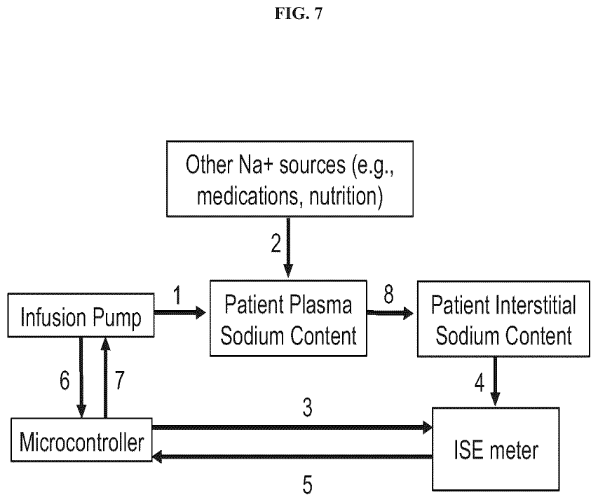

One of the main barriers to a closed-loop sodium administration system is the lack of an established, continuous, biological sodium sensor. Wearable sensors that can detect the sodium content in sweat, saliva, and tears are used in research and distributed commercially, but the sodium secretion in these fluids can vary independent of plasma sodium concentration. Subcutaneous, interstitial fluid sodium concentration measurement is a more reliable alternative, and subcutaneous electrochemical sensors are already established for glucose measurement in closed-loop insulin administration devices. Although plasma and serum glucose concentrations have been shown to equilibrate within 10 minutes, the lag in sodium equilibration is not well-described. However, both glucose and sodium undergo facilitated diffusion from capillary endothelium to the interstitial space; therefore, it is reasonable to hypothesize that sodium, like glucose will equilibrate within 10 minutes. A wearable Microneedle-based extended gate transistor for real-time detection of sodium in interstitial fluids is recently reported, which can be adapted for use in the present invention. In an embodiment, sodium sensor may be placed in the subcutaneous space and measures interstitial sodium concentration. As shown in FIG. 7, the infusion pump adds HTS to the patient's intravascular space (1), increasing the plasma sodium content. Water and sodium added to the intravascular space in intravenous fluid or medication infusions and absorbed enterally also change the patient's plasma sodium concentration (2). By diffusion, the interstitial sodium concentration changes proportionally to the plasma sodium concentration (8). The controller/control unit 13 sends the sodium measurement command to the ISE meter (3), which measures the voltage from a sodium ISE dwelling in the patient's interstitial space (4). The controller/control unit 13 reads the voltage measurement from the ISE meter (5) and converts the voltage to sodium concentration measurement based on previous calibration. The controller/control unit 13 then calculates the cumulative volume of HTS administered based on a revolution counter in the infusion pump (6). Finally, controller/control unit 13 calculates a new rate for HTS infusion and sends a command the infusion pump based on a treatment algorithm to change the operation of the infusion pump, such as adjusting the rate of HTS infusion (7). The sodium sensor may be anchored within the patient by a subcutaneously mounted and easily accessed connector fitting having means for coupling or relaying sensor signals to the infusion pump. A preferred connector fitting has a generally cylindrical configuration adapted for convenient mounting beneath the patient's skin at a proximal end of a catheter leading to a selected in vivo sensor site. The sodium sensor comprises an elongated sensor cable for placement through the connector fitting, and catheter to position a sensor tip at a distal end of the cable substantially at the sensor site. A proximal end of the sensor cable seats within the connector fitting and includes means such as contacts or the like for coupling with the connector fitting so that the connector fitting provides means for electrically coupling the sodium sensor, control unit, and the pump system.

Alternatively, a sodium sensor capable of non-invasive continuous monitoring of plasma or interstitial sodium concentration measurement can be used in this system. In yet another embodiment, the sodium sensor is a continuous sodium sensor based on near-infrared spectroscopy. Near-infrared spectroscopy has already been used to noninvasively measure pH in tissue in vivo and electrolytes including sodium in whole blood in vitro. The major benefit of near-infrared spectroscopy is that it is noninvasive, which would make the closed-loop system easier to use in a variety of situations, especially in pre-hospital care.

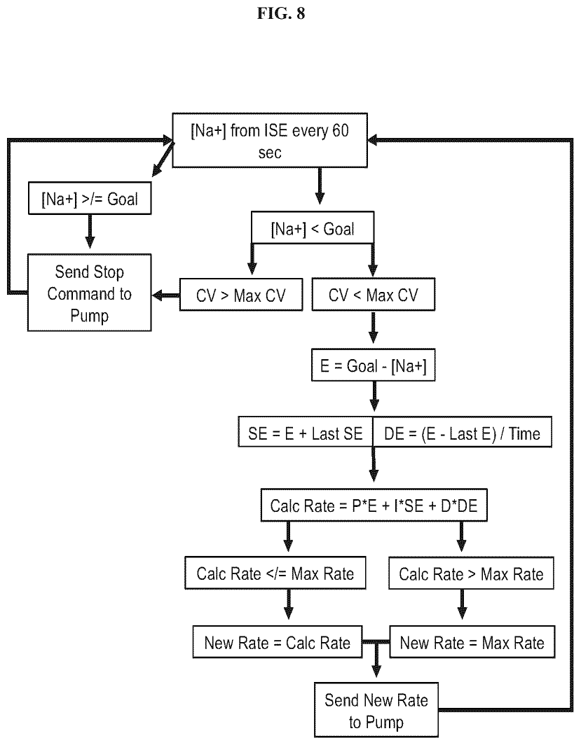

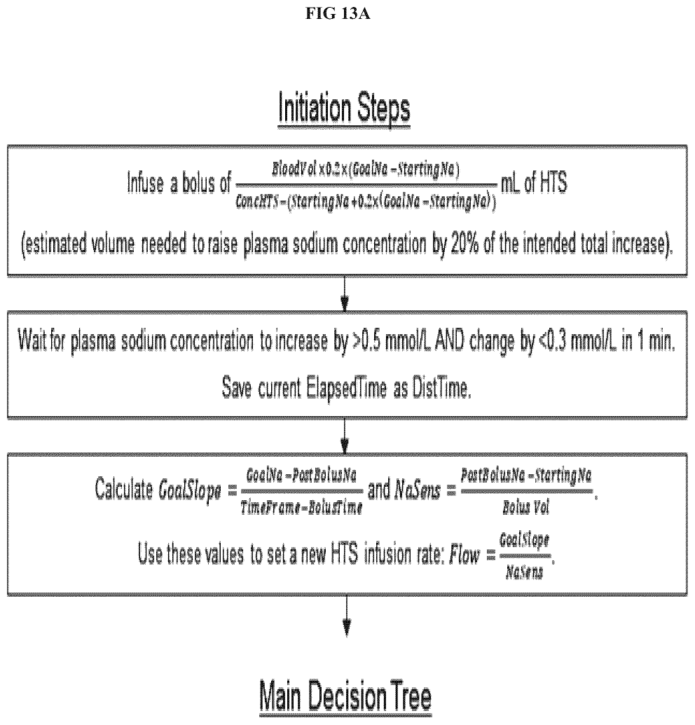

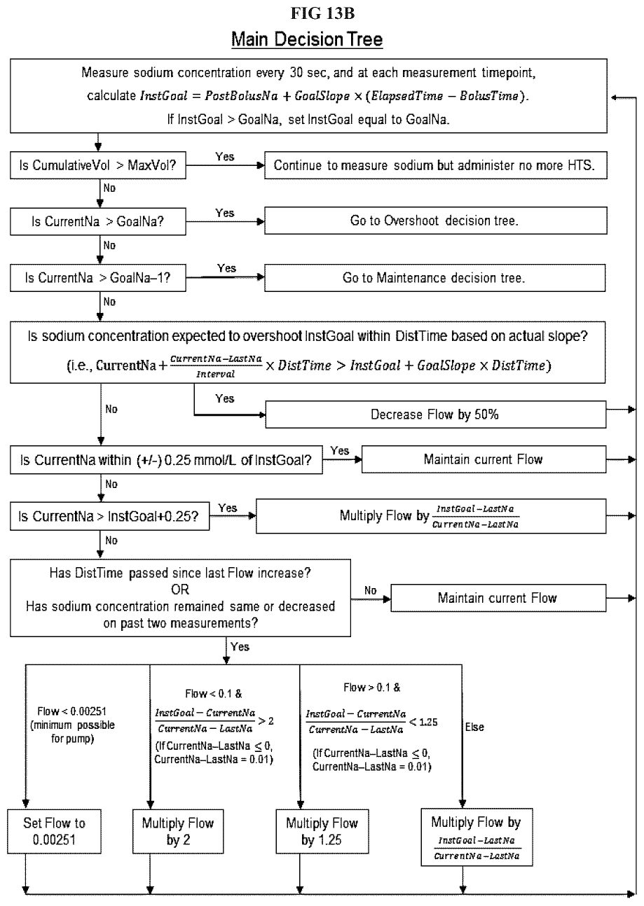

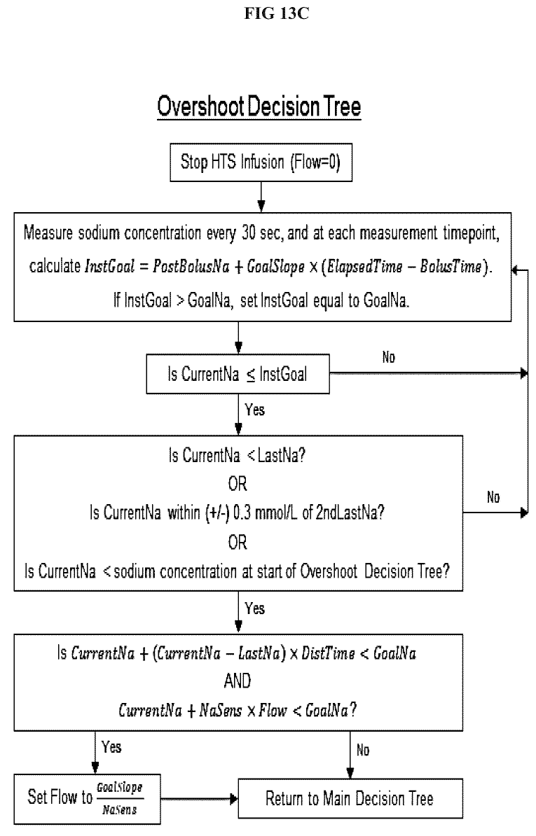

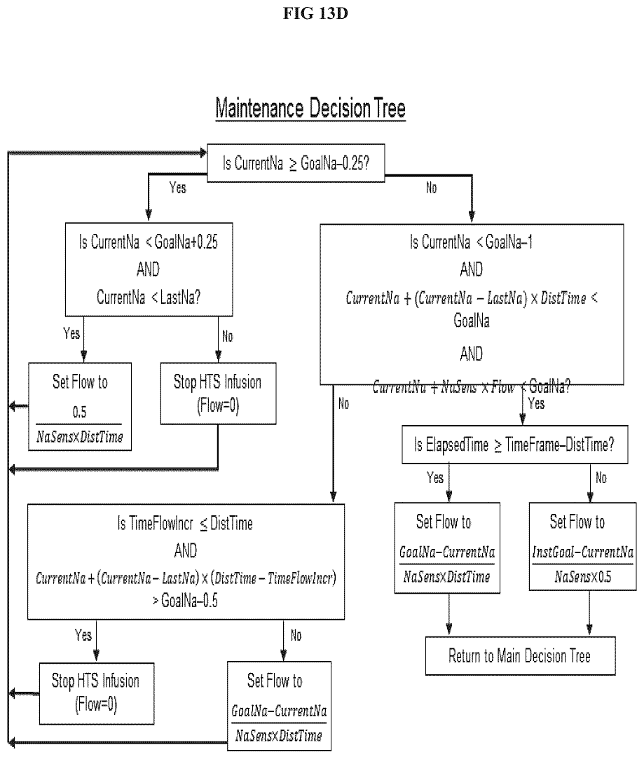

FIG. 8 and FIG. 13A-D show the treatment algorithm used for calculating the rate of hypertonic saline (HTS) infusion using a prototype. The patient's plasma or interstitial sodium concentration ([Na+]) is read from the ISE meter every 30-60 seconds. If [Na+] is greater than or equal to the set goal sodium concentration, the controller/control unit 13 sends a command making the infusion pump stop. If [Na+] is less than the set goal, the controller/control unit 13 compares the cumulative volume administered (based on the infusion pump revolution counter) to the maximum safe HTS dose. If the cumulative volume has met or exceeded the maximum, the controller/control unit 13 send the stop command to the infusion pump. Otherwise, the controller/control unit 13 calculates a new rate for HTS infusion based on the newly measured [Na+]. In the proportion-integral-derivative version of the algorithm shown in FIG. 8, the new HTS infusion rate is determined with the equation:

R=0.002×Eᵢ+0.001×ΣE+0.05×ΔE

where R is the hypertonic saline infusion rate, Ei is the current error as defined by the difference between the goal sodium concentration and the current sodium concentration, ΣE is the sum of the current error and all previous errors since starting the infusion, and ΔE is the difference between the current error and the preceding error.

The difference between the goal and current [Na+] is the Error (E). The sum of errors (SE) is a running total of the error calculated in each iteration. The change in error (DE) is the difference between the current error and the last error divided by the elapsed time between the [Na+] measurements. The E, SE, and DE terms are multiplied by the proportional (P), integral (I), and derivative (D) coefficients, respectively, to determine the calculated rate (Cale Rate). The coefficients vary based on the magnitude of error to avoid overshoot. For example, if the error is less than 5 mmol/L, then P and I decrease by 50%, and D doubles. If the Cale Rate is less than or equal to the maximum safe rate of HTS infusion (Max Rate), the Cale Rate is the New Rate that will be sent to the infusion pump. If Cale Rate exceeds the Max Rate, the New Rate will be equal to the Max Rate. The controller/control unit 13 send a command to the infusion pump to change the motor speed to match the New Rate. A new [Na+] is measured every 60 seconds, and the algorithm is repeated for each new [Na+].

Goal sodium concentration may be set by the user at approximately 140-150 mmol/L. In a more aggressive therapy, goal sodium concentration may be set at approximately 150-155 mmol/L. Very occasionally, a clinician may set goal sodium concentration at approximately 155-160 mmol/L, but never more than 160 mmol/L. The controller will have a default maximum HTS infusion rate of 3 mL/kg/hour and maximum allowed volume of 500 mL 3% saline (or equivalent) in a 4-hour period, but these parameters can be adjusted by the supervising physician based on individual patient needs.

An example of more detailed treatment algorithm used for in vivo administration of HTS is provided in FIG. 13A-D, which is used in testing using a Rat model as described in example 3.

In operation, first demonstrated in a prototype system, the user enters the patient/subject weight, maximum dose, and desired plasma sodium concentration into the firmware, which is then sent to the controller/control unit 13 (A) via a variety of connections, such as the USB serial port (B), which powers the device, and can also log data to a computer. A separate continuous sodium measurement device sends analog signal to an analog-digital converter, and the resulting digital value is used as the input in the closed-loop algorithm. In the PID version of the algorithm shown in FIG. 8, the PID coefficients can be adjusted to account for differences between individual patients and different desired time periods to reach the desired plasma sodium concentration. In the more generalizable form of the treatment algorithm, shown in FIG. 13A-D, the organism's estimated blood volume, starting sodium concentration, goal sodium concentration, and HTS concentration are entered, and the algorithm determines an initial HTS infusion rate and then adjusts as necessary. The algorithm output is the rate in mL/hour of HTS administration, which is sent to the infusion pump.

In the prototype system, this output can be in the form of ASCII commands for infusion pumps with serial communication capabilities, pulse-width-modulation (PWM) for infusion pumps with analog motors, or frequency of a 50% duty cycle square wave for stepper motors. The output signal is sent via one of the pins from the controller/control unit 13 to a separate infusion pump. An aspect of the invention is a stand-alone controller equipped with HTS treatment algorithm as shown in FIG. 8 or FIG. 13A-D, which is used in combination with an infusion pump and a sodium sensor, as in the prototype, capable of automatically adjusts the infusion rate of HTS, based on real-time sodium concentration measurements received from the sodium sensor at a predetermined sampling rate. In alternative design, the controller equipped with such HTS administration algorithm is an integrated part of the infusion pump.

Yet another aspect of this invention is the HTS administration algorithm used by a standalone controller used in combination with an infusing pump or by an integrated controller of an infusing pump. The example treatment algorithms as demonstrated in FIG. 8 and FIG. 13-A-D are used to automatically administer HTS to a patient based on real-time plasma sodium concentration measurement of the patient.

HTS infusion rate was kept at constant rate for the duration of the timeframe based on Equation 1.

$$HTS Infusion Rate = BWV * \frac{{Conc}_{G o a l} - {Conc}_{Starting}}{{Conc}_{H T S} - {Conc}_{G o a l}} * \frac{1}{Timeframe} \qquad ( 1 )$$

In Equation 1, HTS Infusion Rate is the desired rate of HTS infusion in mL/min, BWV is body water volume in mL, Conc_{Goal} is the goal sodium concentration in mmol/L, Conc_{Starting} is the starting sodium concentration in mmol/L, Conc_{HTS} is the sodium concentration in HTS (513 mmol/L), and Time frame is the desired time in min over which to adjust the adjust the sodium concentration. Equation 1 was derived based on the expected volume needed to increase the sodium concentration assuming the HTS distributes evenly throughout the body's water compartments. A formula modified from the literature for calculating rat body water volume is expressed in Equation 2.

BWV=0.5*Mass*1000 (2)

In Equation 2, BWV is the body water volume in mL, and Mass is the rat's mass in kg. After recognizing that the estimated amount of fluid needed to raise a rat's sodium concentration by 10 mmol/L was around 15 mL/kg, the proportion 0.5, which is slightly lower than that reported in the literature, was chosen as a starting point for body water volume estimation to avoid overloading the rat with intravenous fluid. There was a planned interim analysis to determine if the first two control rats achieved the goal sodium concentration despite choosing a slightly low proportion for body water volume estimation. The formula for human body water volume can be similarly deduced and may not be equal to the proportion of 0.5.

The detailed embodiment of the HTS administration algorithm is shown in FIG. 13A-D. FIG. 13A shows logic diagram for the algorithm that sets the initial rate for HTS administration. FIG. 13B shows the main decision tree for adjusting the rate of HTS administration (rate of infusion) to reach the goal sodium concentration. FIG. 13C shows the decision tree for overshoot algorithm, which is used when real sodium concentration measurement exceeds the goal plasma sodium concentration. FIG. 13D is the maintenance decision tree, which demonstrate the algorithm used for maintaining a stable sodium concentration when patient plasma sodium concentration reaches the goal sodium concentration. Closed-loop sodium administration offers advantages over traditional HTS administration: The closed-loop technique will allow for safer and more effective use of HTS for cerebral edema and intracranial hypertension from brain injuries or for hyponatremia. It will be more effective by more reliably increasing the plasma sodium concentration to the desired goal in a short time period. It will be safer by preventing excess administration of HTS and increase of the plasma sodium concentration beyond the therapeutic window. This could avoid complications like metabolic acidosis and osmotic demyelination syndrome. The invention improves current HTS treatment by condensing time consuming and labor-intensive treatment steps into seconds, allowing real-time, automatic adjustments of HTS infusion. The closed-loop treatment algorithm could be integrated into an infusion pump system. However, the invention is more versatile as a stand-alone controller, which can be paired with a variety of sodium measuring devices or infusion pumps.

### Example 1: Continuous Sodium Measurements Using Electrical Conductivity Meter

In a proof-of-concept model for closed-loop sodium administration, we use an electrical conductivity meter and regression equation for continuous sodium concentration calculations.

### 1A: Control Program Development

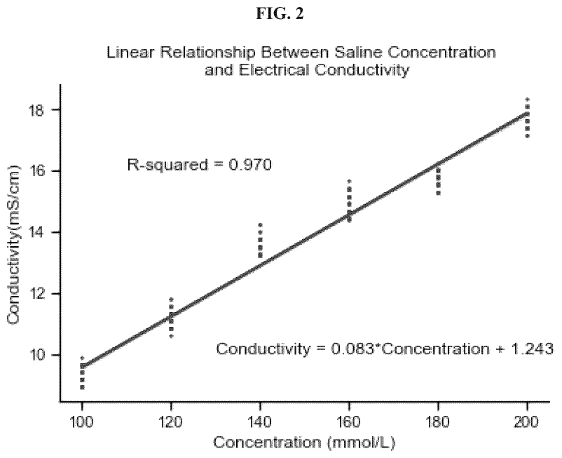

Control program was written in the Arduino Integrated Development Environment (IDE) for adjustment of saline concentration to a specified target with proportional-integral-derivative (PID) control. The program uses adaptive tuning parameters, which increase the rate of hypertonic saline (HTS) injection when the sodium concentration is far from the target and reduce the rate of HTS injection when the sodium concentration is near the target. The infusion stops when the sodium concentration target is reached, and resumes if the sodium concentration then falls below the target. The program was developed on an Arduino Uno prototyping board (Somerville, MA). An aqueous electrical conductivity meter (DFRobot, Shanghai, model no. DFR0300-H, cell constant K=I0) was used to measure the electrical conductivity of saline. Initial calibration data was collected to verify a linear relationship between the saline concentration and electrical conductivity, and a linear regression was fit using Python software (Beaverton, OR) to estimate the electrical conductivity of the target sodium concentration, as shown in FIG. 2.

1B. Testing Program in Benchtop Model with Saline

Syringe pumps made from electronic stepper motors and 3D-printed parts were used to inject and aspirate saline from a beaker of saline to raise the saline concentration while maintaining a constant volume of 200 mL. In developing the control program, the electrical conductivity was used as the input, and the output was the speed of the stepper motors. Coefficients for the proportion, integral, and derivative, are set respectively at: P=4, I=0.1, and D=1. When the measured sodium concentration is within 3 mmol/L of the goal concentration, the coefficients are switched to P=2, I=0.02, and D=0.2 for finer adjustments.

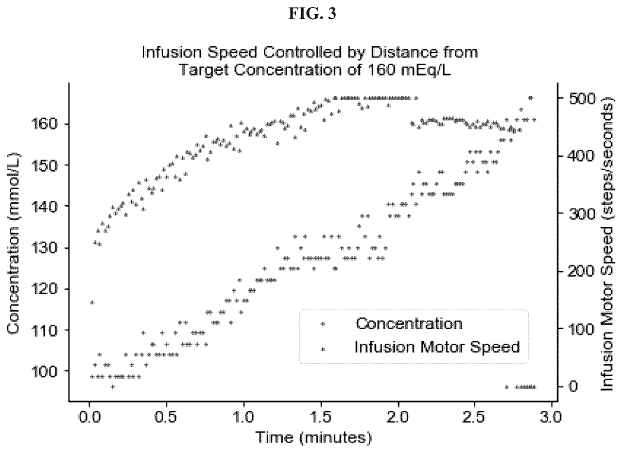

FIG. 3 shows the relationship between saline concentration in the beaker and the infusion motor speed, as controlled by the PID program. The program successfully increased the saline concentration in the beaker to 160 mmol/L and then turned the pumps off while continuing to measure saline concentration. Given that our program successfully adjusted sodium concentration based on real-time measurements in a stable, benchtop system, the next step was testing the system with a more selective sensor in in vitro and in vivo models.

### Example 2: Testing the Sodium Control Program in an In Vitro Circulatory Model with a Sodium Ion Selective Electrode (ISE)

### 2A. Assembling the In Vitro Circulatory Model

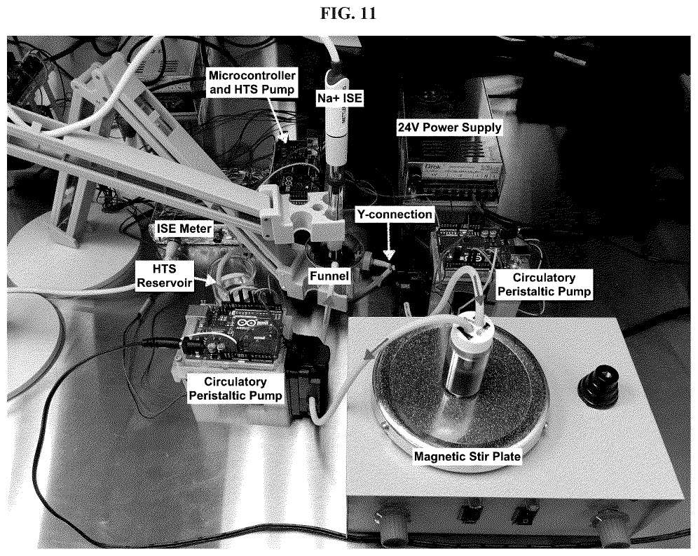

As shown in FIG. 11, a 30-cm length of rubber peristaltic pump tubing with inner diameter of 0.6-mm was connected to a 50-mL funnel stem. The tubing was through a peristaltic pump to a 22-mL capsule with magnetic stir rod on a stir plate. Another 30-cm length of tubing was routed from the capsule on the stir plate back to the opening of the funnel. A glass sodium ISE combined with a reference electrode (Mettler-Toledo, Columbus, Ohio) was positioned in the funnel opening, near the bottom without touching the funnel. A Y-connector was inserted in the tubing between the funnel stem, and the peristaltic pump such that tubing from another peristaltic pump could infuse HTS. The circuit was filled with abattoir-derived porcine whole blood (Innovative Research, Novi, MI). The magnetic stir plate was turned on low to mix the HTS and blood in the capsule, and the peristaltic pump conveyed the blood and HTS through the circuit at 5 mL/min. A constant level of fluid in the funnel was ensured, such that the measurement portion of the ISE was appropriately submerged.

### 2B. Applying the Closed-Loop Sodium Administration Program In Vitro

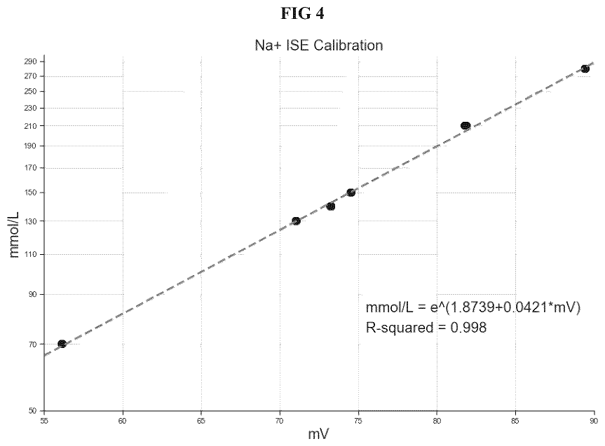

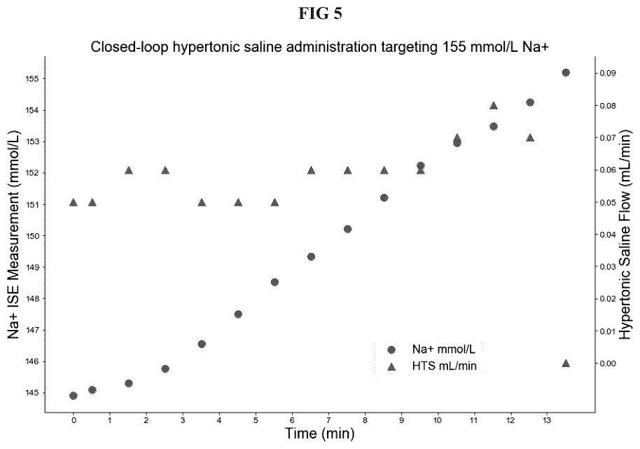

The control program described in Example 1 was adapted to receive input from the sodium ISE described in Section 2A and provide output to the stepper motor driver in the HTS infusion pump. The program was set to measure the plasma sodium concentration at baseline and then every 60 seconds. For each sodium measurement, the controller used the PID control program described in Example 1 to achieve a sodium concentration of 155 mmol/L. Multiple iterations were performed to dial in the tuning coefficients in the PID control program until the sodium concentration in the circuit continuously increased to the goal without overshooting. Once the PID coefficients were determined, the program was run with a starting plasma sodium concentration between 140 and 145 mmol/L, and data for the plasma sodium concentration and HTS infusion rate were recorded at each minute of the experiment. A log-linear regression for ISE calibration resulted in an R-squared value of 0.998, and the calibration equation mmol/L=e\hat{ }(1.8739+0.0421*mV). The calibration plot is shown in FIG. 4. After repeated iterations with trial-and-error adjustments of the PID coefficients, the final coefficients were P=0.002, 1=0.001, and D=0.05. With these PID coefficients, the system effectively controlled the rate of HTS infusion such that there was a gradual increased in the measured plasma sodium concentration until it reached the predetermined goal of 155 mmol/L, at which point the infusion was stopped, as shown in FIG. 5.

### Example 3: Test the Closed-Loop Sodium Administration System in a Rat Model

Testing and tuning the closed loop sodium administration system in a biological model allows us to determine the correlation and temporal relationship between plasma and interstitial sodium changes, which is not well-established. Rats are the chosen animal model for the proof-of-concept experiment because there is established literature for sodium homeostasis research in rats as well for the effect of hypertonic saline in rat models of TBI and stroke.

### 3A. Methods

### (a) Ion-Selective Electrode (ISE) Preliminary Testing and Calibration

A standard, glass combined sodium ISE and reference electrode (Mettler-Toledo, Columbus, OH), a 2.5-mm diameter tubular PVC sodium ISE and reference electrode (NT Sensors, El Catllar, Spain), and a 2.3-mm inner-diameter flow-through PVC sodium ISE and reference electrode (EDT directION, Dover, UK) were all tested for baseline characteristics. Each sensor was conditioned according to manufacturer recommendations prior to use. Each sensor was integrated in a circuit with sodium chloride in DI water (140 mmol/L sodium) flowing past the sensor. The solution was conveyed through 1.6-mm inner-diameter peristaltic pump tubing. The glass electrode was suspended in the center of a 5-cm diameter borosilicate glass funnel in which saline flowed into the wide opening and out through the stem. Paraffin film was placed tightly over the funnel opening to minimize evaporation. The 2.5-mm tubular ISE and reference electrode were housed in a custom, 3-D printed capsule with O-ring-sealed electrode entry points and Luer taper ports on each side for tubing connections. The capsule was designed with 3D modeling software (Fusion 360, Autodesk, San Rafael, CA) and printed with a stereolithography printer and biocompatible resin (FormLabs, Somerville, MA). For the flow-through ISE, the manufacturer-provided housing was used. The saline flow was set to 2.5 mL/min using an adjustable speed peristaltic pump (Boxer, Ottobeuren, Germany) to match the expected mean flow of the rat iliac artery.¹ ISE mV readings were recorded every 30 seconds with an Arduino MEGA 2560 microcontroller-based development board (Arduino, Somerville, MA) and compatible ISE meter (IMACIMUS, NT Sensors, El Catllar, Spain). The test was continued for at least 4 hours for each ISE, and the measurement drift and standard deviation for each ISE were compared.

The flow-through sodium ISE was calibrated with standard solutions of sodium chloride in ultrapure water, ranging from 90-190 mmol/L in increments of 20 mL/L and flowing through the ISE at 2.5 mL/min. Six measurements in m V were recorded for each standard concentration, and a calibration regression was fit (see Data Analysis section).

### (b) Closed-Loop Infusion Algorithm Development

The Arduino Software (Arduino IDE) and Arduino MEGA 2560 were used to program a microcontroller to take measurements from the ISE meter via the Arduino's serial pins every 30 seconds. The flow-through sodium ISE was connected to the ISE meter with a BNC cable. The calculated regression equation was included in the Arduino program to convert m V measurements to sodium concentration in mmol/L. The converted sodium concentration values were used in an algorithm to adjust the speed of the Boxer peristaltic pump which pumped 3% sodium chloride in DI water (HTS) from a flask. Timer1 on the Arduino's ATMEGA microcontroller was used to generate a variable frequency, 50% duty cycle pulse-width-modulation (PWM) waveform. This waveform was sent to the step pin on the peristaltic pump stepper motor driver, and thus, the pump speed was controlled by changing the PWM frequency. The program was designed to allow the user to use the computer keyboard to enter into the Arduino serial monitor a goal sodium concentration and timeframe (time in minutes over which to adjust the sodium concentration). A button was wired to microcontroller interrupt pin which was programmed such that pressing the button would initiate the infusion program, allowing the user to wait for the sodium concentration measurement to equilibrate before starting the infusion. The microcontroller was programmed to perform the following functions upon initiation:
1. Start a timer.
2. Record the starting sodium concentration.
3. Calculate the estimated volume of HTS required to increase the sodium concentration to the goal.
4. Administer 20% of the estimated volume of HTS at the max bolus rate of 2 mL/kg/hr.
5. Record sodium concentration measurements from the ISE meter every 30 seconds and wait for the sodium concentration to stabilize.
6. After the sodium concentration stabilizes, calculate the HTS sensitivity (change in sodium concentration per mL of HTS administered) and the distribution time (time from administration of bolus to stabilization of sodium concentration).
7. Use the HTS sensitivity to set a new infusion rate that will increase the sodium concentration to goal over the amount of time remaining in the timeframe.
8. With each new ISE measurement:
9. Calculate the instantaneous slope (actual change in sodium concentration over the last measurement interval).
10. Calculate instantaneous goal sodium concentration using the time elapsed and ideal slope (difference between goal and starting sodium concentrations divided by the timeframe).
11. If the sodium concentration has reached the goal, start a low maintenance rate to keep the concentration at the goal.
12. Otherwise, if the sodium concentration will exceed the goal within the distribution time based on the instantaneous slope, decrease the HTS infusion rate.
13. Otherwise, compare the current sodium concentration to the instantaneous goal and increase or decrease the HTS infusion rate accordingly.

An in vitro circuit was used to test and adjust the closed-loop infusion algorithm. A 30-mL beaker was placed on a stir-plate with low magnetic stirring. One 20-cm length of 1.6-mm inner-diameter peristaltic tubing extended from the beaker to the flow-through sodium electrode. Another 20-cm length of tubing extending from the opposite end of the flow-through electrode, through the peristaltic pump, and into the beaker. Then, 12-30 mL of sodium chloride in DI water solution ranging from 130-150 mmol/L sodium was placed in the beaker. The range of volume were based on the estimated blood volume of a 200-500 g rat, and the sodium concentration range was based on the expected physiologic blood sodium concentration. Developing the algorithm over a range of volumes and starting sodium concentrations was expected to make it more versatile. A trial-and-error method was used to adjust the algorithm parameters such that the actual change in sodium concentration matched the desired change in sodium concentration as closely as possible. The adjusted parameters included:
Acceptable change from one ISE measurement to the next for the concentration to be considered stable.
Increase in HTS infusion rate when the sodium concentration was less than the instantaneous goal.
Decrease in HTS infusion rate when the sodium concentration exceeded the instantaneous goal.
Decrease in HTS infusion rate when the sodium concentration was expected to exceed the goal within the distribution time based on instantaneous slope
Decrease in HTS infusion rate when the sodium concentration reached the goal.
Increase in HTS infusion rate if the sodium concentration fell below goal after having reached it.

For each change in the algorithm, the in vitro trial was reiterated. If the change led to an obvious problem early in the trial, the trial would be stopped to fix the problem in the algorithm before trying again. If the algorithm was expected to successfully increase the sodium concentration to goal within the timeframe or within several minutes of the end of the timeframe, the trial was completed. Qualitative assessment of the trend of sodium concentrations was used to determine when the algorithm was adequate for quantitative testing in the rat experiments.

The full logic diagrams of the algorithm used for the Evaluation of the Closed-Loop System in a Rat model is shown in FIG. 13A-D.

### Acronyms Used in FIG. 13A-D

BloodVol=total blood volume (mL) of the organism; for rats,
BloodVol=0.06×M assx I 000
ConcHTS=sodium concentration (mmol/L) in the hypertonic saline (HTS) being administered
GoalNa=intended final plasma sodium concentration (mmol/L)
StartingNa=plasma sodium concentration (mmol/L) measured before initial bolus of HTS
CurrentNa=most recently measured plasma sodium concentration (mmol/L)
TimeFrame=intended time (min) for the plasma sodium concentration to increase from initial to goal
ElapsedTime=time (min) since starting the initial bolus
DistTime=distribution time (min) for initial bolus and subsequent stabilization of plasma sodium concentration
PostBolusNa=plasma sodium concentration (mmol/L) after the initial bolus
Goal Slope=intended rate of change (mmol/L/min) in plasma sodium concentration
NaSens=sodium sensitivity (mmol/L/mL); increase in plasma sodium concentration per mL of HTS
InstGoal=intended sodium concentration at any given time based on the GoalSlope and elapsed time
Flow=HTS infusion rate (mL/min)
Cumulative Vol=cumulative volume (mL) of HTS administered since the start of the initial bolus
Max Vol=maximum allowable volume (mL) of HTS to administer
LastNa=previous sodium concentration (mmol/L) measurement
2ndLastNa=penultimate sodium concentration (mmol/L) measurement
Interval=time (min) between CurrentNa and LastNa measurements
TimeFlowincr=time (min) since last increase in Flow

### Notes

If the calculated HTS infusion rate (Flow, mL/min) exceeds the predetermined maximum safe infusion rate (2 mL/kg/hr for rats in the experiments), then the Flow is changed to the maximum before being sent to the infusion pump.

If the calculated HTS infusion rate (Flow, mL/min) is less than the lowest possible for the infusion pump (0.00251 mL/min for the pumps in the experiments), then the Flow is changed to zero, stopping the infusion.

### (c) Evaluation of the Closed-Loop System in a Rat Model

Sodium measurement and HTS administration apparatus: The Arduino MEGA 2560 was wired to a peristaltic pump and ISE meter as described above. A feature was added to the Arduino program to allow one-point calibrations by drawing blood samples, measuring the sodium concentration with a bench-top analyzer (pHOx Plus, Nova Biomedical, Waltham, MA), and entering the value into the Arduino serial monitor. Internal two-point calibrations and quality-control functions were performed on the bench-top analyzer at the beginning of each day of experiments.

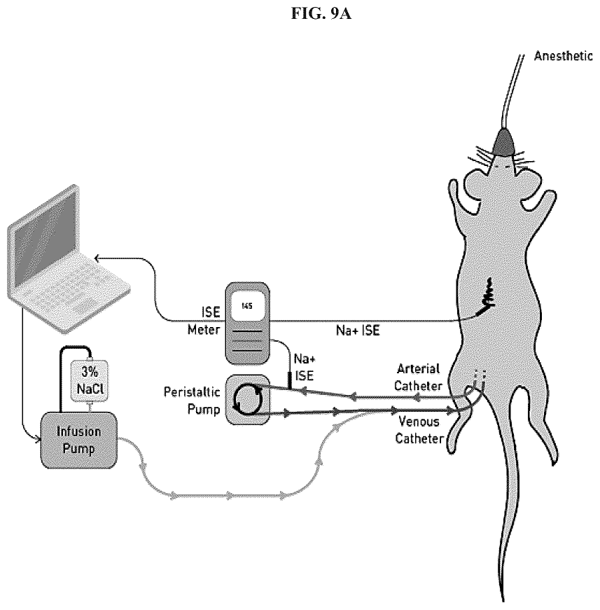

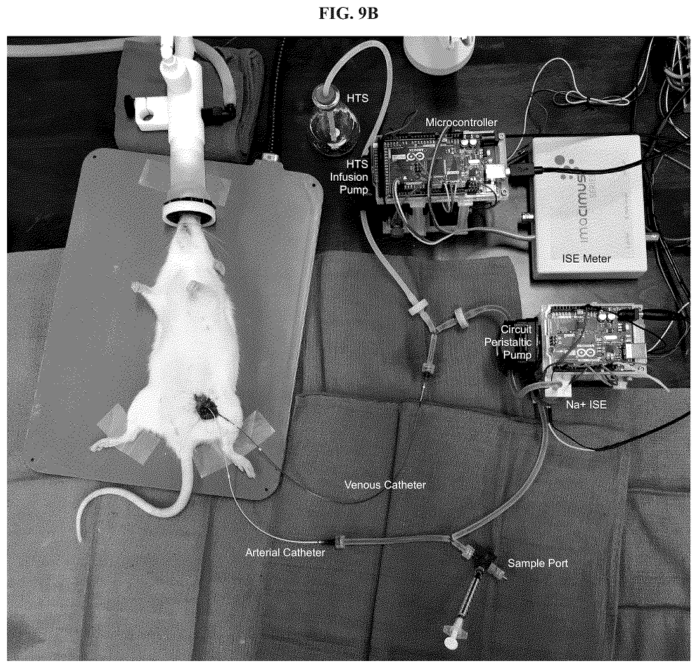

To set up the circuit for blood circulation through the ISE and HTS infusion, a second, independent peristaltic pump was placed several cm away from the Arduino MEGA 2560. On the negative-pressure side of the peristaltic pump, a length of peristaltic pump tubing, which would convey blood from the rat's body, was connected to the gravity-dependent side of the vertically-oriented flow-through sodium ISE and reference electrode. The vertical orientation aided with bubble clearance. Proximal to the ISE, the length of tubing had a Y-connector with an attached stop-cock for blood sample collection without flow interruption. Distal to the ISE, there was another length of tubing which passed through the rollers of the blood-flow peristaltic pump and terminated at a Y-connector on the positive-pressure side of the peristaltic pump. A solution of 3% sodium chloride in DI water (HTS) was sterilized with a microfilter and contained in a 50-mL flask with a stopper. A length of peristaltic tubing passing from the HTS reservoir through a hole in the stopper, then through the rollers of the HTS peristaltic pump, and terminated at the Y-connector on the positive-pressure side of the blood-flow peristaltic pump. A final length of tubing was connected to the remaining arm of this Y-connector, and this is where mixing of blood and HTS would take place on the way back to the rat. All connections were made with barbed and Luer taper connectors. The experimental apparatus is shown in FIG. 9.

All tubing, vessels, and components that would come in contact with rat blood were sanitized with 70% isopropyl alcohol and rinsed with sterile, ultrapure water with the exception of the flow-through electrodes which were thoroughly rinse with sterile, ultrapure water. Full sterilization was not required because non-survival surgeries were performed. The HTS tubing was primed with HTS up to the Y-connecting, and the remaining tubing was primed with lactated Ringer's solution.

Subjects and creation of extracorporeal shunt: The University of Texas Health San Antonio Institutional Animal Care and Use Committee protocols were followed for all animal care and procedures. Sprague-Dawley rats (6 male and 6 female) were obtained at 226-250 g (corresponding to 6-8 weeks of age for males and 11-13 weeks of age for females) from Charles River Laboratory (Kingston, NY). They were given free access to food and water and allowed to acclimate for at least one week. Rats were anesthetized with 1-2% isoflurane inhaled via a nose cone. Appropriate depth of anesthesia was ensured by depth and pattern of respiration and pedal reflex testing. Femoral artery and vein cannulas (SAi Infusion Technologies, Lake Villa, IL) were primed with heparinized lactated Ringer's solution (100 U/mL). Each rat was positioned supine, and an incision was created over the femoral vessels. Segments of the femoral artery and femoral vein 1-2 cm in length were dissected out, and the distal ends were tied off with silk suture. A loop of silk suture was placed around the proximal side of each vessel segment and suspended to temporarily impede blood flow. Micro scissors were used create a partial opening in the vessel, and a cannula was inserted a few cm. The suture loop was then tied over the vessel and cannula to secure the cannula in the vessel. Once the venous cannula was in place, the rat was administered an IV bolus of 500 U/kg heparin. Then, the arterial cannula was placed. The arterial cannula was connected to the negative pressure end of the peristaltic pump circuit, and the venous cannula was connected to the positive pressure end of the peristaltic circuit. Then, the blood-flow peristaltic pump was started at 2.5 mL/min to approximate the expected mean rat iliac artery blood flow.

Measurement of plasma sodium concentration and administration of HTS: Each rat's mass in kg was entered in the program code, and the program was uploaded to the Arduino MEGA 2560, which started recording ISE sodium measurements every 30 seconds. The measurements were visible in real time in a table in the Arduino serial monitor output. After initiating blood flow through the extracorporeal circuit, there was baseline period of at least 10 minutes to allow for plasma sodium stabilization and ISE membrane stabilization. During this period, blood sample was drawn from the stop-cock and run on the benchtop analyzer. For each blood sample, 0.15 mL blood was withdrawn from stop-cock and discarded to prevent sample contamination from stagnant blood. Then, an additional 0.15 mL blood was withdrawn in a new syringe and aspirated directly from the syringe by the benchtop analyzer. This process was repeated every 5 to 10 min throughout the experiments for one-point recalibration of the ISE. After at least 10 minutes, one or more recalibrations, and three consecutive ISE sodium measurements within 0.5 mmol/L of each other, the HTS infusion could be started. The goal sodium concentration 10 mmol/L greater than the starting sodium concentration (rounding to the nearest whole number) was chosen and entered into the Arduino serial monitor. For some rats, the sodium concentration changed by up to 3 mmol/L between entering the goal sodium and starting the HTS infusion. Therefore, some goal sodium concentrations were more or less than 10 mmol/L above the ultimate starting sodium concentration. A timeframe of 20 min for reaching the goal sodium was entered for each rat. Then, the start button was pressed to initiate the HTS infusion. For rats in the experimental group, HTS infusion rate was determined by the closed-loop algorithm described. For rats in the control group, HTS infusion rate was kept at constant rate for the duration of the timeframe based on Equation 1.

$$HTS Infusion Rate = BWV * \frac{{Conc}_{G o a l} - {Conc}_{Starting}}{{Conc}_{H T S} - {Conc}_{G o a l}} * \frac{1}{Timeframe} \qquad ( 1 )$$

In Equation 1, HTS Infusion Rate is the desired rate of HTS infusion in mL/min, BWV is body water volume in mL, Conc_{Goal} is the goal sodium concentration in mmol/L, Conc_{Starting} is the starting sodium concentration in mmol/L, Conc_{HRS} is the sodium concentration in HTS (513 mmol/L), and Time frame is the desired time in min over which to adjust the adjust the sodium concentration. Equation 1 was derived based on the expected volume needed to increase the sodium concentration assuming the HTS distributes evenly throughout the body's water compartments. A formula modified from the literature for calculating rat body water volume is expressed in Equation 2.^{2, 3}

BWV=0.5*Mass*1000 (2)

In Equation 2, BWV is the body water volume in mL, and Mass is the rat's mass in kg. After recognizing that the estimated amount of fluid needed to raise a rat's sodium concentration by 10 mmol/L was around 15 mL/kg, the proportion 0.5, which is slightly lower than that reported in the literature, was chosen as a starting point for body water volume estimation to avoid overloading the rat with intravenous fluid. There was a planned interim analysis to determine if the first two control rats achieved the goal sodium concentration despite choosing a slightly low proportion for body water volume estimation.

Although the HTS infusion stopped after 20 min in the control group, the HTS infusion could continue past 20 min in the experimental group, as determined by the algorithm, to reach and maintain the goal sodium. The rats were kept under anesthesia and connected to the infusion. After that, if there was no cavitation during blood sample draws and the rat's respiratory pattern was stable, the experiment was continued up to 60 min after the start of the HTS infusion. At the end of each experiment, the cannulas were removed, and the rat was euthanized by decapitation after ensuring adequate depth of anesthesia. Data were saved from the Arduino serial monitor output.

### 3B. Data Analysis

For preliminary ISE testing, the drift was calculated by subtracting the starting

mV value from the final m V value during a period of continuously increasing or continuously decreasing reading up to 4 hours. The difference was divided by the elapsed time in min. Standard deviation of the m V values was calculated in Excel (Microsoft, Seattle, WA). Using the Excel Data Analysis add-in, a log-linear regression was calculated for ISE calibration with the natural log of the concentration as the dependent variable and ISE m V as the independent variable.

The MatPlotLib and Pandas libraries in Python (Python Software Foundation, Beaverton, OR) were used to plot the sodium concentration and HTS infusion rate time courses for the in vitro and rat experiments. For the rat experiments, the main outcome measure was the sum of errors for the actual vs. intended sodium concentrations. The intended sodium concentration was defined as a linear increase from starting sodium concentration to goal sodium concentration over 20 min from the start of the HTS infusion, and the goal sodium concentration thereafter. The sum of errors was calculated as the sum of the absolute values of the difference between intended sodium concentration and ISE-measured sodium concentration at each time point. The sodium concentrations at 40 min after the start of the HTS infusion and the cumulative HTS volume were compared between the control and experimental groups to determine whether any difference in the sum of errors could be attributable to a systematic difference in the volume of HTS administered. Due to the small sample size and non-Normal distribution of the data, the Kruskall-Wallis U-test was used for significance testing. The SciPy library in Python was used to calculate the Kruskall-Wallis U-test statistics.

### 3C. Results

### (a) Ion-Selective Electrode (ISE) Preliminary Testing

The 2.5-mm tubular PVC ISE had the least predictable drift, ranging from 0.02 to 0.05 m V/min in both directions. It also had the highest standard deviation at 7.3 m V. Next, the glass ISE had the lowest drift (0.004 mV/min) and standard deviation (0.25 mV). However, 8 mL of fluid were required to submerge the sensor, and the circuit configuration was prone to air bubbles and variations in the fluid level in the funnel. Finally, the flow-through ISE had a continuous downward drift of 0.01 mV/min and standard deviation of 0.80 mV, along with the advantage of the flow-through design allowing for minimal additional circuit fluid. This would translated to minimal additional blood outside the rat's native circulation for in vivo experiments. Thus, the flow-through electrode offered the best combination of precision and practicality, and it was used for the remainder of the experiments.

### (b) Closed-Loop Infusion Algorithm Development

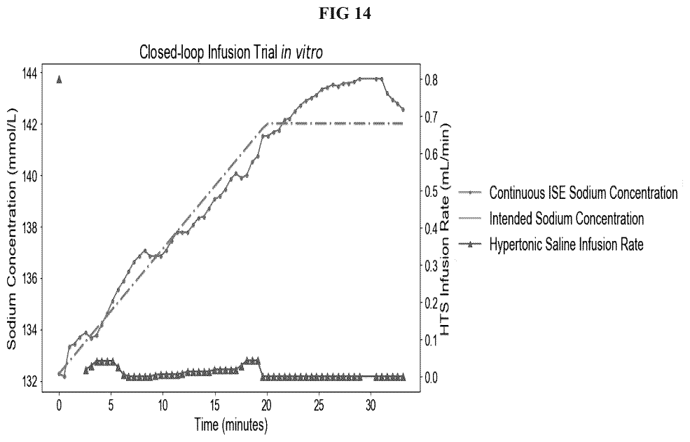

A total of 17 in vitro trials were completed with the flow-through ISE circuit. Many additional trials were terminated early and not saved because the errors that led to early termination were usually code errors with unintended consequences in the algorithm function. An example of a trial with the final version of the algorithm is shown in FIG. 14. The HTS infusion rate was adjusted throughout the trial such that the sodium concentration gradually increased to goal, despite some deviation from the intended slope.

### (c) Evaluation of the Closed-Loop System in a Rat Model

Out of the 12 rats, 11 survived the anesthesia, surgery, and extracorporeal shunt long enough for completion of the experiment with data collection at least 40 minutes after the start of HTS administration. The tenth rat died during HTS administration. The cause of death was not clear, but air or thromboembolism were possible. Table 1 shows the starting plasma sodium concentration (after ISE equilibration and recalibration), final sodium concentrations, and sum of errors from intended sodium concentration for each rat. The interim analysis to assess the volume of HTS administered to control rats was performed after Rat 4, the second control rat. The sodium concentration of each of the first two control rats initially exceeded the goal, and by 40 min after the start of HTS infusion, the sodium concentrations were within 2 mmol/L of the goal. Therefore, the weight-based formula for determining the volume and rate of HTS infusion was not changed for the remaining control rats.

Compared to rats administered the pre-calculated, weight-based, constant infusion of HTS, sodium concentrations in rats administered HTS via a closed-loop infusion program were closer to the intended sodium concentrations over the course of the infusion. The sums of errors in the experimental group were significantly lower than the sums of errors in the control group (Mann-Whitney U-test statistic 2.0, p=0.011). Forty minutes after the start of HTS infusions, the differences between actual and goal sodium concentrations were similar between the experimental and control groups (Mann-Whitney U-test statistic 7.0, p=0.17). Furthermore, the cumulative volume of HTS administered after 40 minutes was similar between the two groups (Mann-Whitney U-test statistic 5, p=0.082), as shown in Table 1.

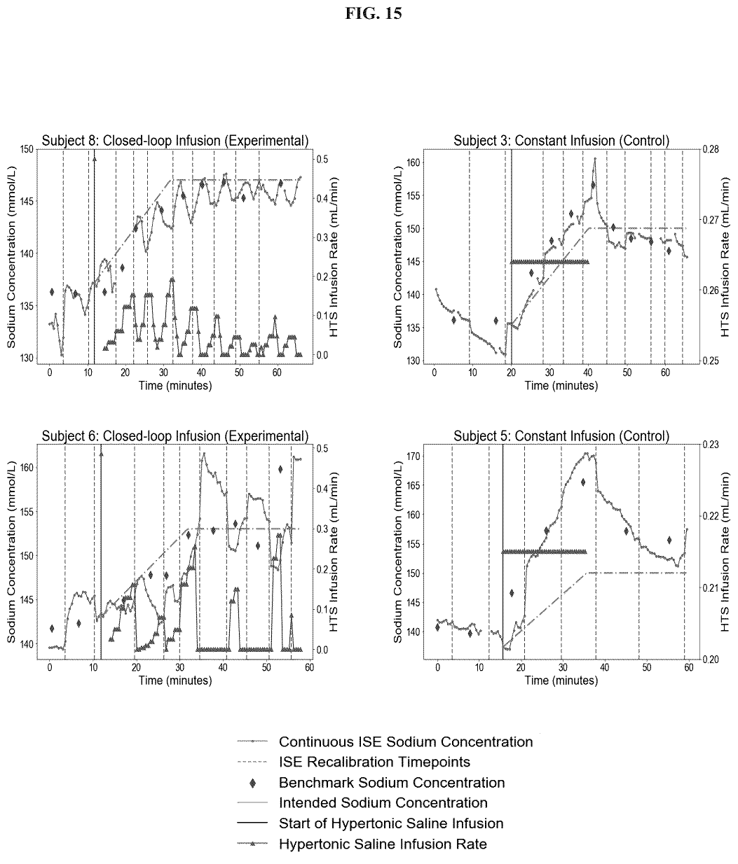

The sodium concentration and HTS infusion rate trends for the rats with the highest and lowest sum of errors in each group are shown in FIG. 18. For the rat with the lowest sum or errors, the sodium concentration increased gradually from starting concentration to goal over 20 min as intended, as shown in the top left panel of FIG. 15. Then, the closed-loop program system continued to periodically administer HTS as needed to maintain the sodium concentration near the goal until 60 min after the start of the HTS infusion, at which point the experiment was stopped. With this rat, the ISE sodium concentrations were very close to the benchtop analyzer's measurements; ISE drift was not a substantial problem here. However, ISE drift was a problem for the rat in the experimental group with the highest sum of errors, as shown in the bottom left panel of FIG. 15. Although many of the benchtop analyzer sodium concentration measurements were close to the intended sodium concentration, the ISE measurements varied substantially from the intended sodium concentration and from the corresponding benchtop analyzer measurements. Thus, each recalibration resulted in a large change in the ISE measurement, and the HTS infusion rate fluctuated between periods of no flow and brief periods of high flow.

ISE drift was also present in the control rats but did not influence the HTS infusion rate after the start of the infusion because it was pre-calculated using the rat mass and starting sodium concentration. As shown in the panels on the right in FIG. 15, control rats' sodium concentration followed a consistent pattern of rising well above the goal within 20 min of the start of the infusion and then gradually descending back to goal. When the control rats were monitored up to 50 min, the sodium concentrations still remained within 2 mmol/L of the goal (Table 1). Thus, the formula for calculating the volume of HTS needed to reach the goal sodium concentration was accurate despite the initial overshoot seen in every rat in the control group.

| | | | | | Sodium | Sodium | Sodium | | | HTS | HTS | |
|---|---|---|---|---|---|---|---|---|---|---|---|---|
| | | | Starting | Goal | at | at | at | Diff. at | Sum of | Vol. at | Vol. at | |
| | | Mass | Sodium | Sodium | 40-min | 45-min | 50-min | 40-min | Errors | 20-min | 40-min | Missing |
| Subject | Group | (kg) | (mmol/L) | (mmol/L) | (mmol/L) | (mmol/L) | (mmol/L) | (mmol/L) | (mmol/L) | (mL/kg) | (mL/kg) | Values |
| 1 | Exp. | 0.277 | 136.0 | 146.0 | 144.4 | NA | NA | 1.6 | 154.1 | 4.4 | 8.9 | 0 |
| 2 | Exp. | 0.262 | 139.7 | 150.0 | 150.1 | 150.4 | NA | 0.1 | 192.4 | 7.4 | 14.0 | 1 |
| 3 | Control | 0.290 | 135.4 | 150.0 | 148.2 | 145.7 | NA | 1.8 | 188.9 | 18.0 | 18.0 | 8 |
| 4 | Control | 0.244 | 138.4 | 149.0 | 150.3 | 149.3 | 147.9 | 1.3 | 478.8 | 15.8 | 15.8 | 3 |
| 5 | Control | 0.246 | 137.3 | 150.0 | 152.5 | NA | NA | 2.5 | 783.5 | 14.2 | 14.2 | 0 |
| 6 | Exp. | 0.243 | 143.5 | 153.0 | 148.6 | 160.9 | NA | 4.4 | 259.4 | 5.0 | 8.5 | 0 |
| 7 | Control | 0.303 | 138.3 | 149.0 | 154.1 | 150.5 | 148.2 | 5.1 | 446.3 | 15.0 | 15.0 | 0 |
| 8 | Exp. | 0.250 | 137.2 | 147.0 | 146.7 | 145.8 | 146.0 | 0.3 | 127.1 | 6.6 | 10.7 | 10 |
| 9 | Exp. | 0.302 | 136.6 | 147.0 | 146.6 | 146.5 | 146.6 | 0.4 | 153.5 | 7.7 | 15.2 | 0 |
| 11 | Control | 0.264 | 134.3 | 145.0 | 145.5 | 146.1 | 144.9 | 0.5 | 318.0 | 14.6 | 14.6 | 0 |
| 12 | Exp. | 0.248 | 136.8 | 147.0 | 146.3 | 148.7 | 146.3 | 0.7 | 178.7 | 5.9 | 11.5 | 0 |

Table 1. Summary of each rat's mass, key sodium concentration values, difference between actual and goal sodium concentration measured at 40 min (Diff. at 40-min), sum of differences between actual and intended sodium concentrations at each measurement timepoint from the start of the HTS infusion through 40 min later (Sum of Errors), cumulative HTS volume administered by 20 min and 40 min (HTS Vol.), and number of sodium measurement values missing during the 40-min period. Sodium values were measured with the flow-through ISE, except for the goal sodium which was chosen prior to starting the HTS infusion. Interpolation was used to replace missing values for the sum or errors calculation. For rats in which the experiment was not continued to 45 or 50 min, the sodium values for those timepoints are marked NA Please note that the timepoints listed in this table are in reference to the start of HTS administration and do not correspond to the timescale in FIG. 15, which starts with a baseline period prior to HTS administration. ISE=ion-selective electrode, HTS=hypertonic saline.

### 3D. Discussion

A closed-loop system for hypertonic saline administration was successfully evaluated in a rat model. To our knowledge, this is the first time it has been demonstrated that HTS infusion can be administered at adjusted rate based continuous measurement of a subject's real-time plasma sodium concentration.

Using a flow-through sodium ISE in an extracorporeal circulatory circuit, the plasma sodium concentration was measured every 30 seconds, and a microcontroller-based development board was used to adjust the HTS infusion rate based on the sodium concentration measurements. Compared to rats given a pre-calculated, weight-based, constant rate infusion of HTS (control group), rats that were administered HTS using the closed-loop system had sodium concentrations more closely resemble the intended sodium concentration course (experimental group). This was not due a systematic difference in the amount of HTS that were administered because the cumulative volume of HTS at 40 min after the start of the infusion was similar between the experimental and control groups. Furthermore, the difference between the measured sodium concentration and goal sodium concentration at 40 min after the start of the infusion was similar between groups. Thus, the method of HTS administration, not the volume of HTS administered, caused the different paths to reach the goal sodium concentration.

The control group was given the weight-based, constant rate infusion for over 20 min. During this time, the sodium concentration measurements of the control rats consistently rose well above the goal (i.e. target sodium concentration) before gradually return to the goal and remain there. The initial overshoot in the control group likely reflects the time needed for the HTS to distribute from the intravascular compartment to the extravascular compartments in the rat. Once the HTS had distributed across the water compartments, the sodium concentration stabilized around the goal sodium concentration. The pattern of sodium concentration in the control rats was fairly similar to the pattern of circulating blood volume after a 10-min intravenous saline infusion in rats performed by Nose et al. In their experiment, the 30% of the infused volume was retained in the intravascular compartment at the end of the 10-min infusion, but only 5-10% of the infused volume was retained in the intravascular compartment 40 min after the end of the infusion. However, Nose et al. did not measure solute retention so direct comparison of time for sodium distribution is not possible.

The initial overshoot of the sodium concentration goal was avoided in the experimental group with the use of closed-loop HTS administration system because real-time sodium concentration measurements allowed the HTS infusion to be slowed at the beginning of the HTS administration to provide time for distribution. Once the sodium concentration was near the goal, the HTS infusion continued at a variable maintenance rate to compensate for HTS continuing to distribute from the intravascular to extravascular compartments.

Approximately 9-16 mL/kg HTS were required to raise the rats' plasma sodium concentrations by 10 mmol/L 40 min after the start of the HTS infusion. One control rat was administered 18 mL/kg HTS, but this was related to an ill-timed change in measured sodium concentration causing the goal sodium concentration to be 15 mmol/L higher the measured sodium concentration at the start of the HTS infusion. On average, the HTS volume administered was higher than previous studies using HTS boluses to examine the effects of HTS on cerebral edema in rats. The change in plasma sodium concentration was not reported in these studies, but Schreibman et al. reported that serum osmolarity increased from 301 to 310 mOsm/kg after 0.7 mL/kg of 23.4% HTS, which has an equivalent sodium content to 5.5 mL/kg of 3% HTS. If the increase in osmolarity was purely from the solute in the HTS, the expected increase in sodium concentration would be 4.5 mmol/L. This change in sodium concentration per unit of HTS is proportional to that seen in our study.

Interpretation of the results would have been aided by measurement of renal sodium clearance. The short time period of the experiment may have mitigated the effects of renal sodium clearance. Hansson et al. reported no change in renal sodium excretion within hours of an intravenous sodium load in spontaneously hypertensive rats and normotensive Wistar-Kyoto rats. However, the rats in their experiment were administered a low-sodium diet for 3 days before the sodium load, and the 1.5-mmol/kg sodium load they gave was much lower than the 5-8 mmol/kg sodium given to the rats in this experiment. Furthermore, Mozaffari et al. found that Wistar-Kyoto and spontaneously hypertensive rats administered 8.6 mmol/kg sodium in the form of intravenous HTS excreted 88-106% of the sodium load within 90 min of the start of the infusion.8 Measuring renal excretion of sodium in our study would have helped quantify the relative contributions of distribution of HTS out of the intravascular space and renal clearance of sodium to reductions in the plasma sodium concentration.

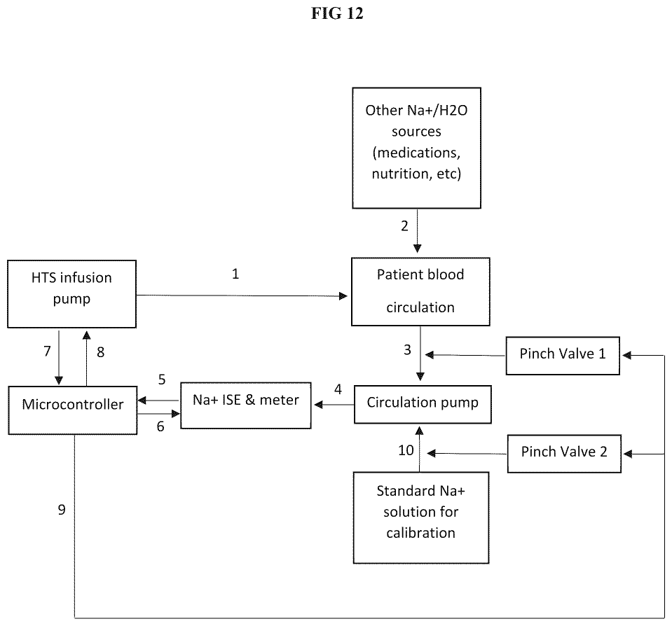

Another limitation of our study was the ISE measurement drift. Frequent recalibration with blood samples measured on the benchtop analyzer allowed for a successful experiment despite the drift. However, some of the experimental subjects' HTS infusion rates were substantially impacted by swings in the sodium concentration due to drift and recalibration, there were delays about 2.5 min between the blood sample draw and recalibration due to the benchtop analyzer processing time. With rapid changes in the plasma sodium concentration, the sodium concentration in some of the blood samples may not have even reflected the actual plasma sodium concentration 2.5 min later. Furthermore, the frequent blood samples reduced the rats circulating blood volume and limited the amount of time the experiment could be continued. It would not be sustainable to draw 0.2 mL blood every 5-10 min for multiple hours without a blood transfusion. A process of internal recalibration with every measurement or every few measurements by running a standard solution through the flow-through ISE has since been added to the device improve the accuracy and sustainability of the plasm sodium concentration measurements (FIG. 12).

FIG. 12 shows the sequence of interactions between the microcontroller, infusion pump, and ion-selective electrode (ISE) meter for in vivo administration of HTS in a rat model. The infusion pump adds hypertonic saline (HTS) to the patient's intravascular space (1), increasing the plasma sodium content. Water and sodium added to the intravascular space in intravenous fluid or medication infusions or absorbed enterally also change the patient's plasma sodium concentration (2). The patient's blood flows through a circulation pump (3), which maintains flow through the ISE (4). The circulation pump speed can be controlled by a rotary encoder to adjust the flow. The microcontroller sends the sodium measurement command to the ISE meter (5), which measures the voltage from a sodium ISE in contact with the patient's blood. The microcontroller reads the voltage measurement from the ISE meter (5) and converts the voltage to sodium concentration based on previous calibration. The microcontroller then calculates the cumulative volume of HTS administered based on a revolution counter in the infusion pump (7). Finally, microcontroller calculates a new rate for HTS infusion and sends a command the infusion pump to change the rate of HTS infusion (8). Every 30 seconds, the ISE measures a new plasma sodium concentration which the microcontroller uses to calculate a new HTS infusion rate. Every 10 minutes, the microcontroller sends a signal to a switch board close Pinch Valve 1 and open Pinch Valve 2 (9). This interrupts circulation of blood through the ISE and allows the circulation pump to move the standard Na+ solution for calibration through the ISE (10). The microcontroller then performs a one-point recalibration, and the pinch valves reset to allow blood to again flow through the ISE. The I 0-minute calibration period may be adjusted as the user desires.

With the internal recalibration process, the closed-loop sodium administration system will be more robust for longer periods of sodium measurement and adjustment, allowing for testing closed-loop sodium administration in rats with cerebral edema, which may take several hours to days to develop after traumatic brain injury or vascular injury. Longer periods of testing would also be helpful for comparing the closed-loop system to longer constant infusions of HTS which may allow for reaching the sodium goal without overshooting. Finally, it would be helpful to test the performance of the closed-loop system with external influences on the sodium concentration such as dietary sodium or simultaneous infusion of hypotonic solutions or in the setting of disorders such as diabetes insipidus or syndrome of inappropriate anti-diuretic hormone.

Finally, the flow-through ISE is not ideal because it requires creation of an extracorporeal circulatory circuit for sodium concentration measurements. A noninvasive or minimally invasive sodium sensor would be better. Some groups have created sensors that measure interstitial sodium concentration, but it is not clear that these sensors would be precise enough.^{11, 12} Furthermore, the concordance between the plasma and interstitial sodium is unknown. Future studies incorporating both a minimally invasive interstitial sodium sensor and the flow-through ISE for plasma sodium concentration measurements would help answer these questions.

### Advantages

The benefit of a closed-loop sodium administration system is potentially immense for malignant cerebral edema treatment. The advantages of continuous, closed-loop control of plasma sodium concentration over the current standard of care does not reside solely in its ability to be safer in controlling osmotic shifts and more effective in treating cerebral edema. It also has the potential to alter the way we manage fluids and sodium in a variety of medical conditions,

Efficient, precise sodium adjustment and fluid resuscitation is especially important in patients with severe hyponatremia or hypovolemia from dehydration or hemorrhage, all of which can coexist with TBI or stroke, particularly in military personnel in austere environments, athletes, refugees, or neglected elderly patients. In such situations, adequate fluid resuscitation must be achieved quickly to prevent further end-organ damage from hypoperfusion, especially in the brain. Furthermore, correcting the hyponatremia can prevent seizures and reduce cerebral edema. The closed-loop sodium administration system would allow clinicians tighter control over sodium concentration during resuscitation to ensure a patient's intravascular volume and sodium homeostasis are restored without delay and without overshooting.

The closed-loop sodium administration system would also expand the settings in which HTS can safely be used. In the pre-hospital setting and austere environments, the need for frequent blood sampling, laboratory services and physician monitoring limits the use of HTS. Although previous trials have not shown a benefit to prehospital HTS for TBI, it is possible that the standardized amounts of HTS administered were subtherapeutic.^{26, 27} Applying a closed-loop sodium administration system in the prehospital setting would allow for personalized volumes of HTS to achieve the necessary plasma osmolality for a therapeutic effect despite the lack of laboratory services and physicians. Regular hospital wards could also benefit because the nurse-to-patient ratio on the ward is usually not enough for the frequent blood draws required for safe HTS administration. The closed-loop sodium administration system could maintain normonatremia or keep hypernatremia from normalizing too rapidly to prevent rebound cerebral edema in patients transferred from the ICU to the regular ward after a TBI or stroke.

Beyond brain injury, closed-loop sodium administration would make a positive impact on fluid management in a wide variety of medical conditions. Hyponatremia is a risk factor for mortality in hospitalized patients, and slow rate of correction has been associated with poor outcomes.^{28, 29} The closed-loop sodium administration system could be used to optimally correct sodium in conditions causing hyponatremia like syndrome of inappropriate anti diuretic hormone (SIADH), cerebral salt wasting, dialysis disequilibrium syndrome, heart failure, or cirrhosis. Using hypotonic fluid such as dextrose 5% in water instead of hypertonic saline, the device could also be used to manage conditions causing hypernatremia such as diabetes insipidus, toxicity from drugs such as lithium, impaired thirst mechanism, or osmotic diuresis (e.g., from mannitol or diabetes mellitus). In patients whose sodium is already maintained at a constant value by the device, alarms could be programmed to give clinicians early warnings of physiology changes. For example, if the HTS required to maintain the target sodium concentration suddenly increases, the clinician would know to look for conditions such as SIADH or cerebral salt wasting. If the HTS required to maintain the target sodium concentration suddenly drops, the clinician would know to look for conditions such as diabetes insipidus. Finally, the device could help with the challenging problem of determining overall fluid status by estimating total body water using the change in plasma sodium concentration for a given volume of fluid with known concentration.

In addition to its direct clinical benefits, a closed-loop sodium administration could improve our understanding of hypertonic saline's effect on cerebral edema and intracranial pressure. Although HTS is recommend for reducing ICP, it is not recommended for improving neurological outcomes, and there is insufficient evidence to recommend continuous infusion over boluses of HTS.² However, these are conditional recommendations based on low-quality evidence.² Results from previous studies, especially in humans, are confounded by variation in sodium concentration over time and variable changes in plasma sodium concentration for a given amount of HTS. Standardizing the increase in plasma sodium concentration in a group of animal subjects or human participants would help to control these confounding factors. Furthermore, the continuous sodium concentration measurement would greatly increase the resolution of the data. Continuous intracranial pressure measurement from an ICP monitor could be compared in real-time to the sodium concentration rather than snapshots of the sodium concentration every few hours. Such data could help in determining the ideal dosing and timing of HTS (i.e., ideal target sodium concentration, prophylactic administration vs. reactive to changes in ICP, and bolus vs. continuous infusions).

### REFERENCES

1. Juul N, Morris G F, Marshall S B, Marshall L F. Intracranial hypertension and cerebral perfusion pressure: influence on neurological deterioration and outcome in severe head injury. Journal of Neurosurgery. 2000; 92 (1):1-6. doi: 10.3171/jns.2000.92.1.0001
2. Cook A M, Morgan Jones G, Hawryluk G W J, et al. Guidelines for the Acute Treatment of Cerebral Edema in Neurocritical Care Patients. Neurocritical Care. 2020; 32 (3):647-666. doi: 10.1007/s12028-020-00959-7
3. George J C, Zafar W, Bucaloiu I D, Chang A R. Risk Factors and Outcomes of Rapid Correction of Severe Hyponatremia. Clinical Journal of the American Society of Nephrology. 2018; 13 (7):984-992. doi: 10.2215/CJN.13061117
4. Riha H M P, Erdman M J P, Vandigo J E M, et al. Impact of Moderate Hyperchloremia on Clinical Outcomes in Intracerebral Hemorrhage Patients Treated With Continuous Infusion Hypertonic Saline: A Pilot Study. Critical Care Medicine. 2017; 45 (9). doi:10.1097/CCM.0000000000002522
5. Maguigan K L, Dennis B M, Hamblin S E, Guillamondegui O D. Method of Hypertonic Saline Administration: Effects on Osmolality in Traumatic Brain Injury Patients. Journal of Clinical Neuroscience. 2017; 39:147-150. doi: 10.1016/j.jocn.2017.01.025
6. Webster D L, Fei L, Falcone R A, Kaplan J M. Higher-volume hypertonic saline and increased thrombotic risk in pediatric traumatic brain injury. Journal of Critical Care. 2015; 30 (6):1267-1271. doi: 10.1016/j.jcrc.2015.07.022
7. TBI-related Emergency Department (ED) Visits|Concussion|Traumatic Brain Injury|CDC Injury Center. Published Mar. 29, 2019. Accessed Apr. 1, 2020. https://www.cdc.gov/traumaticbraininjury/data/tbi-ed-visits.html
8. Dewan M C, Rattani A, Gupta S, et al. Estimating the global incidence of traumatic brain injury. Journal of Neurosurgery. 2018; 130 (4):1080-1097. doi: 10.3171/2017.10.JNS17352
9. Rubiano A M, Carney N, Chesnut R, Puyana J C. Global neurotrauma research challenges and opportunities. Nature. 2015; 527 (7578): S193-S197. doi:10.1038/nature16035
10. Schreibman D L, Hong C M, Keledjian K, et al. Mannitol and hypertonic saline reduce swelling and modulate inflammatory markers in a rat model of intracerebral hemorrhage. Neurocrit Care. 2018; 29 (2):253-263. doi:10.1007/s12028-018-0535-7
11. Mangat H S, Wu X, Gerber L M, et al. Hypertonic Saline is Superior to Mannitol for the Combined Effect on Intracranial Pressure and Cerebral Perfusion Pressure Burdens in Patients With Severe Traumatic Brain Injury. Neurosurgery. Published online Mar. 16, 2019. doi:10.1093/neuros/nyz046
12. Yin J, Zhang H, Chen H, Lv Q, Jin X. Hypertonic Saline Alleviates Brain Edema After Traumatic Brain Injury via Downregulation of Aquaporin 4 in Rats. Med Sci Monit. 2018; 24:1863-1870. doi:10.12659/msm.907212
13. Scalfani M T, Dhar R, Zazulia A R, Videen T O, Diringer M N. Effect of osmotic agents on regional cerebral blood flow in traumatic brain injury. Journal of Critical Care. 2012; 27 (5):526.e7-526.e12. doi:10.1016/j.jcrc.2011.10.008
14. Zeng H K, Wang Q S, Deng Y Y, et al. Hypertonic saline ameliorates cerebral edema through downregulation of aquaporin-4 expression in the astrocytes. Neuroscience. 2010; 166 (3):878-885. doi: 10.1016/j.neuroscience.2009.12.076
15. Elliott M B, Jallo J J, Barbe M F, Tuma R F. Hypertonic saline attenuates tissue loss and astrocyte hypertrophy in a model of traumatic brain injury. Brain Res. 2009; 1305:183-191. doi:10.1016/j.brainres.2009.09.104
16. Soustiel J F, Vlodavsky E, Zaaroor M. Relative effects of mannitol and hypertonic saline on calpain activity, apoptosis and polymorphonuclear infiltration in traumatic focal brain injury. Brain Research. 2006; 1101 (1):136-144. doi:10.1016/j.brainres.2006.05.045
17. Garvin R. Neurocritical Care Protocol for Hypertonic Saline. :3.
18. Boughton C K, Hovorka R. Is an artificial pancreas (closed-loop system) for Type 1 diabetes effective? Diabetic Medicine. 2019; 36 (3):279-286. doi:10.1111/dme.13816
19. Kong E, Nicolaou N, Vizcaychipi M P. Hemodynamic stability of closed-loop anesthesia systems: a systematic review. Minerva Anestesiologica. 2020; 86 (1). doi:10.23736/S0375-9393.19.13927-2
20. Cuartero M, Parrilla M, Crespo G. Wearable Potentiometric Sensors for Medical Applications. Sensors. 2019; 19 (2):363. doi:10.3390/s19020363
21. Buono M J, Claros R, DeBoer T, Wong J. Na+ secretion rate increases proportionally more than the Na+ reabsorption rate with increases in sweat rate. Journal of Applied Physiology. 2008; 105 (4):1044-1048. doi:10.1152/japplphysiol.90503.2008
22. Dubach J M, Lim E, Zhang N, Francis K P, Clark H. In vivo sodium concentration continuously monitored with fluorescent sensors. Integrative Biology. 2011; 3 (2):142-148. doi:10.1039/c0ib00020e
23. Mostafalu P, Akbari M, Alberti K A, Xu Q, Khademhosseini A, Sonkusale S R. A toolkit of thread-based microfluidics, sensors, and electronics for 3D tissue embedding for medical diagnostics. Microsyst Nanoeng. 2016; 2. doi:10.1038/micronano.2016.39
24. Chen C, Zhao X-L, Li Z-H, Zhu Z-G, Qian S-H, Flewitt A. Current and Emerging Technology for Continuous Glucose Monitoring. Sensors. 2017; 17 (12):182. doi: 10.3390/s17010182
25. Boyne M S, Silver D M, Kaplan J, Saudek C D. Timing of Changes in Interstitial and Venous Blood Glucose Measured With a Continuous Subcutaneous Glucose Sensor. Diabetes. 2003; 52 (11):2790-2794. doi:10.2337/diabetes.52.11.2790
26. Werner S L. Prehospital hypertonic saline resuscitation of patients with hypotension and severe traumatic brain injury: A randomized controlled trial. Annals of Emergency Medicine. 2005; 45 (2):229-230. doi:10.1016/j.annemergmed.2004.10.027
27. Bulger E M, May S, Brasel K J, et al. Out-of-Hospital Hypertonic Resuscitation Following Severe Traumatic Brain Injury: A Randomized Controlled Trial. JAMA. 2010; 304 (13):1455-1464. doi:10.1001/jama.2010.1405
28. Giordano M, Ciarambino T, Priore E L, et al. Serum sodium correction rate and the outcome in severe hyponatremia. The American Journal of Emergency Medicine. 2017; 35 (11):1691-1694. doi:10.1016/j.ajem.2017.05.050
29. Nzerue C M, Baffoe-Bonnie H, You W, Falana B, Dai S. PREDICTORS OF OUTCOME IN HOSPITALIZED PATIENTS WITH SEVERE HYPONATREMIA. JOURNAL OF THE NATIONAL MEDICAL ASSOCIATION. 2003; 95 (5):9.
30. Sweet C S, Emmert S E, Seymour A A, Stabilito I I, Oppenheimer L. Measurement of cardiac output in anesthetized rats by dye dilution using a fiberoptic catheter. J Pharmacol Methods. 1987; 17 (3):189-203. doi:10.1016/0160-5402 (87) 90050-7
31. Nose H, Sugimoto E, Morimoto T, Usui S, Aomi T. Continuous recording of plasma sodium concentration and blood volume in awake rats. Jpn J Physiol. 1986; 36 (3):607-611. doi:10.2170/jjphysiol.36.607
32. Wiig H, Luft F C, Titze J M. The interstitium conducts extrarenal storage of sodium and represents a third compartment essential for extracellular volume and blood pressure homeostasis. Acta Physiologica. 2018; 222 (3):e13006. doi:10.1111/apha.13006
33. Cserr H F. Role of secretion and bulk flow of brain interstitial fluid in brain volume regulation. Ann N Y Acad Sci. 1988; 529:9-20. doi:10.1111/j.1749-6632.1988.tb51415.x
34. Verbalis J G. Brain volume regulation in response to changes in osmolality. Neuroscience. 2010; 168 (4):862-870. doi:10.1016/j.neuroscience.2010.03.042
35. Weissmann M, Dar D, Krausz M. Whole blood and hypertonic saline resuscitation improve the outcomes of controlled hemorrhagic shock combined with penetrating brain injury in rat. bioRxiv. Published online Oct. 19, 2018. doi:10.1101/448266
36. Leibowitz A, Brotfain E, Koyfman L, et al. Treatment of combined traumatic brain injury and hemorrhagic shock with fractionated blood products versus fresh whole blood in a rat model. European Journal of Trauma and Emergency Surgery. 2019; 45 (2):263-271. doi:10.1007/s00068-018-0908-9
37. Brensilver J M, Daniels F H, Lefavour G S, et al. Effect of variations in dietary sodium intake on sodium excretion in mature rats. Kidney International. 1985; 27 (3):497-502. doi:10.1038/ki. 1985.38
38. Heidrich J P, Niemeier A, Seyfarth M. Continuous analysis in extracorporeally circulating blood—a rat model applying flow-through ion-selective electrodes for the measurement of Ca2+, K+, Na+ and pH. Clin Chem Lab Med. 1998; 36 (11):847-854. doi:10.1515/CCLM.1998.149
39. Kerby J D, Sainz J G, Zhang F, et al. Resuscitation from hemorrhagic shock with HBOC-201 in the setting of traumatic brain injury. Shock. 2007; 27 (6):652-656. doi:10.1097/01.shk.0000248584.10400.dc
40. Glober N K, Sprague S, Ahmad S, et al. Acetazolamide Treatment Prevents Redistribution of Astrocyte Aquaporin 4 after Murine Traumatic Brain Injury. Neurosci J. 2019; 2019. doi: 10.1155/2019/2831501
41. Ellerby G E C, Smith C P, Zou F, Scott P, Soller B R. Validation of a spectroscopic sensor for the continuous, noninvasive measurement of muscle oxygen saturation and pH. Physiological Measurement. 2013; 34 (8):859-871. doi:10.1088/0967-3334/34/8/859
42. Soller B R, Favreau J, Idwasi P O. Investigation of Electrolyte Measurement in Diluted Whole Blood Using Spectroscopic and Chemometric Methods. Appl Spectrosc. 2003; 57 (2):146-151. doi:10.1366/000370203321535042
43. Zheng Y B, Omar R., Zhang R, Tang N., et al, A Wearable Microneedle-Based Extended Gate Transistor for Real-Time Detection of Sodium in Interstitial Fluids. Advance Material. 2021 2021 Dec. 16; e2108607. doi:10.1002/adma.202108607
44. Mu J Y, Hansson G C, Bergström G, Lundgren O. Renal sodium excretion after oral or intravenous sodium loading in sodium-deprived normotensive and spontaneously hypertensive rats. Acta Physiologica Scandinavica. 1995; 153 (2):169-177. doi:10.1111/j.1748-1716.1995.tb09848.x
45. Mozaffari M S, Roysommuti S, Shao Z H, Wyss J M. Renal Responses to Hypertonic Saline Infusion in Salt-Sensitive Spontaneously Hypertensive Rats. The American Journal of the Medical Sciences. 1997; 314 (6):370-376. doi:10.1016/S0002-9629 (15) 40247-2

1. An infusion system for administering hypertonic saline (HTS) to a patient, the infusion system comprising:
a) an infusion device for delivering HTS to a patient;
b) at least one sensor for continuous monitoring of plasma sodium concentration of the patient, wherein the sensor takes measurements of plasma sodium concentration of the patient at a predetermined sampling frequency; and
c) a controller that sets the sampling frequency of said sensor, receives said measurements of the patient's plasma sodium concentration from the sensor, and automatically adjusts the rate of HTS administration of the infusion device based on the real time measurement of the patient's plasma sodium concentration using a treatment algorithm.

2. The infusion system according to claim 1, wherein the infusion device delivers HTS to the patient at a calculated infusion rate based on plasma sodium concentration measurement using a treatment algorithm, which may include a proportion-integral-derivative (PID) equation, wherein the calculated rate of HTS administration cannot exceed a maximum rate of 500 mL 3% saline (or equivalent) per hour.

3. The infusion system according to claim 2, wherein the infusion device automatically suspends HTS delivery when patent's plasma sodium concentration is greater than or equal to said goal sodium concentration or when a cumulative administered volume is greater than or equal to a maximum safe dose.

4. The infusion system according to claim 3, wherein the infusion device automatically resumes HTS delivery when measured plasma sodium concentration is below the goal sodium concentration and cumulative administered volume is below maximum safe dose.

5. The infusion system according to claim 2, wherein the predefined goal sodium concentration is set at approximately 140-160 mmol/liter by the physician based on individual patient need.

6. The infusion system according to claim 1, wherein the infusion system further includes one or more alarms to warn about bubbles in the infusion, and/or empty infusion reservoir.

8. The infusion system according to claim 3, maximum safe dose of cumulative administered volume is 500 mL 3% saline (or equivalent) in a 4-hour period.

9. The infusion system according to claim 2, wherein proportional-integral-derivative (PID) equation is:
R=0.002×Eᵢ+0.001×ΣE+0.05×ΔE
where R is the hypertonic saline infusion rate, Ei is the current error as defined by the difference between the goal sodium concentration and the current sodium concentration, IE is the sum of the current error and all previous errors since starting the infusion, and L′.1E is the difference between the current error and the preceding error.

10. The infusion system for treating cerebral edema using HTS, comprising
a) an implantable sodium sensor for in vivo monitoring of a patient plasma sodium concentration;
b) an implantable connector fitting for supporting said implantable sodium sensor within the patient to permit transcutaneous access to said implantable sodium sensor for removal and replacement without removing said connector fitting from the patient;
c) a controller coupled by said fitting to said sensor for generating a signal based on the monitored patient plasma sodium concentration using a treatment algorithm; and
c) a pump for administering HTS stored therein to the patient, said pump including means responsive to said signal from said controller to administer the HTS at adjusted rate.

11. The infusion system of claim 1, wherein said sensor is ion selective electrode (ISE) optode, sodium-selective optode, near-infrared spectrometer or miscroneedle-based extended gate transistor, wherein the sensor is accurate within 1 mmol/L in the range of 120-170 mmol/L and have a sampling rate of 60 seconds or less.

12. A method to treat cerebral edema, comprising:
a) automatically infusing HTS into the patient at an infusion rate;
b) continuously measuring plasma sodium concentration of a patient at a predetermined sampling rate;
c) comparing measured patient's plasma sodium concentration to a goal plasma sodium concentration;
d) calculating a new infusion rate based on measured patient's plasma sodium concentration using a Proportion-Integral-Derivative equation or a treatment algorithm;
e) adjusting the infusion rate to the calculated infusion rate if
i) the measured sodium concentration is below said goal plasma sodium concentration; and
ii) the calculated infusion rate is less than a maximum infusion rate; and
f) repeating steps a) to e) while the measured sodium concentration is less than the goal sodium concentration;
wherein automatic HTS infusion is temporarily suspended when a cumulative infusion volume to said patient is greater or equal to a maximum safe infusion dose or when measured sodium concentration is equal to or greater than goal sodium concentration; and automatic HTS infusion resumes when measured plasma sodium concentration is below the goal plasma sodium concentration and cumulative administered volume is below maximum safe dose.

13. The method of claim 12, wherein said maximum safe infusion dose is 500 mL 3% saline (or equivalent) in a 4-hour period.

14. The method of claim 12, wherein said goal plasma sodium concentration is set at approximately 140-160 mmol/liter by a physician based on individual patient's need.

15. The method of claim 12, wherein said maximum infusion rate is 3 mL/kg/hour.
